(19) United States
(12) Patent Application Publication
(10) Pub. No.: US 2024/0287572 A1
(43) Pub. Date: Aug. 29, 2024

(54) METHODS, COMPOSITIONS, AND KITS FOR DETECTING HYDROLASE ENZYME ACTIVITY

(71) Applicant: MESO SCALE TECHNOLOGIES, LLC., Rockville, MD (US)

(72) Inventors: John H. KENTEN, Arlington, VA (US); Jacob N. WOHLSTADTER, Palm Beach Gardens, FL (US)

(21) Appl. No.: 18/555,331

(22) PCT Filed: Apr. 15, 2022
(86) PCT No.: PCT/US2022/025015

§ 371 Date: Oct. 13, 2023

Related U.S. Application Data
(60) Provisional application No. 63/175,946, filed on Apr. 16, 2021.

(51) Int. Cl. C12Q 1/34; C12Q 1/682

(52) U.S. Cl. CPC C12Q 1/34; C12Q 1/682

(57) ABSTRACT

The invention relates to methods, compositions, and kits for detection of hydrolase enzyme activity in a sample.

### FIELD OF THE INVENTION

The invention relates to methods, compositions, and kits for detection of hydrolase enzyme activity in samples.

### BACKGROUND

Highly sensitive and specific detection of hydrolase enzyme activity has many applications in diagnosing and monitoring diseases, drug development and customized medicine. Assays for detecting enzyme activity typically require detecting a change in concentration of a substrate, which use methods that are susceptible to higher signal-to-noise ratios. The ability to detect hydrolase enzyme activity with high sensitivity will lead to more accurate diagnosis and more accurate development of drugs that act as enzyme inhibitors or activators.

### SUMMARY OF THE INVENTION

In embodiments, the disclosure provides a method for detecting a hydrolase enzyme in a sample, comprising: (a) contacting the sample with an oligonucleotide detection reagent, wherein the oligonucleotide detection reagent comprises: (i) a targeting agent complement (TAC): (ii) a targeting agent blocker that is complementary to at least a portion of the TAC; and (iii) a hydrolase cleavage site: wherein the TAC and the targeting agent blocker are hybridized, wherein the hydrolase cleaves the oligonucleotide detection reagent at the hydrolase cleavage site, thereby (i) destabilizing hybridization of the TAC and the targeting agent blocker and (ii) generating an unblocked oligonucleotide comprising the TAC; and (b) detecting the unblocked oligonucleotide, thereby detecting the hydrolase enzyme in the sample.

In embodiments of the method, the TAC and the targeting agent blocker are on a contiguous strand of the oligonucleotide detection reagent. In embodiments, the oligonucleotide detection reagent further comprises a detectable label. In embodiments, the oligonucleotide detection reagent comprises, in 5′ to 3′ order, the targeting agent blocker, the hydrolase cleavage site, the TAC, and the detectable label.

In embodiments of the method, the TAC is on a first strand of the oligonucleotide detection reagent, and the targeting agent blocker and the hydrolase cleavage site are on a second strand of the oligonucleotide detection reagent. In embodiments, the targeting agent blocker comprises a first region and a second region, wherein the hydrolase cleavage site is positioned between the first region and the second region; and wherein the first region and the second region of the targeting agent blocker hybridize to first and second regions of the TAC on the second strand. In embodiments, the TAC, the hydrolase cleavage site and the targeting agent blocker are comprised on a single strand.

In embodiments of the method, the hydrolase enzyme is a nuclease and the hydrolase cleavage site comprises a nucleic acid that is cleaved by the nuclease. In embodiments, the hydrolase enzyme is a peptidase and the hydrolase cleavage site comprises a peptide that is cleaved by the peptidase. In embodiments, the hydrolase enzyme is a glycoside hydrolase and the hydrolase cleavage site comprises a polysaccharide that is cleaved by the glycoside hydrolase.

In embodiments, the method further comprises, prior to step (b), immobilizing the unblocked oligonucleotide to a detection surface comprising a targeting agent, wherein the targeting agent is a binding partner of the TAC, wherein uncleaved oligonucleotide detection reagent does not substantially bind to the detection surface. In embodiments, detecting the unblocked oligonucleotide comprises detecting if the unblocked oligonucleotide is bound to the detection surface. In embodiments, detecting the unblocked oligonucleotide comprises: (i) using the unblocked oligonucleotide as a primer in an extension reaction to form an extension product; and (ii) detecting the extension product. In embodiments, the extension product is detected by hybridizing the extension product to a complementary sequence comprising a detectable label. In embodiments, the extension product is detected by hybridization to an array.

In embodiments of the method, hybridization of the TAC with the targeting agent blocker substantially prevents binding of the TAC to the targeting agent on the detection surface.

In embodiments of the method, the TAC and the targeting agent comprise complementary oligonucleotides, and the immobilizing comprises hybridizing the TAC of the unblocked oligonucleotide to the targeting agent on the detection surface.

In embodiments of the method, the detectable label is detected by using light scattering, optical absorbance, fluorescence, chemiluminescence, electrochemiluminescence (ECL), bioluminescence, phosphorescence, radioactivity, magnetic field, or combinations thereof. In embodiments, the detectable label is an electrochemiluminescent label, and the detecting comprises measuring an ECL signal.

In embodiments of the method, the sample is contacted with one or more additional copies of the oligonucleotide detection reagent.

In embodiments, the method comprises immobilizing the plurality of unblocked oligonucleotides to the detection surface and detecting the plurality of unblocked oligonucleotides.

In embodiments of the method, the detection surface comprises a particle. In embodiments, the detection surface comprises a well of a multi-well plate. In embodiments, the detection surface comprises an electrode.

In embodiments, the method is a multiplexed method for detecting one or more hydrolase enzymes in a sample, wherein step (a) comprises contacting the sample with a plurality of oligonucleotide detection reagents, wherein each oligonucleotide detection reagent comprises a TAC, a targeting agent blocker hybridized to the TAC, a hydrolase cleavage site, and a detectable label, wherein, for each hydrolase enzyme in the sample, a hydrolase cleaves an oligonucleotide detection reagent comprising a unique hydrolase cleavage site for the hydrolase, thereby generating a plurality of unblocked oligonucleotides, wherein each unblocked oligonucleotide comprises a unique TAC: wherein, prior to step (b), the plurality of unblocked oligonucleotides are immobilized to a detection surface comprising a plurality of targeting agents, wherein each targeting agent is a binding partner of a unique TAC, and wherein uncleaved oligonucleotide detection reagent does not substantially bind to the detection surface; and wherein step (b) comprises detecting the plurality of unblocked oligonucleotides bound to the detection surface, thereby detecting the one or more hydrolase enzymes in in the sample. In embodiments, each unique TAC comprises a unique oligonucleotide sequence. In embodiments, each hydrolase enzyme is a nuclease and the hydrolase cleavage site of each oligonucleotide detection reagent comprises a nucleic acid. In embodiments, each hydrolase enzyme is a peptidase and the hydrolase cleavage site of each oligonucleotide detection reagent comprises a peptide. In embodiments, each hydrolase enzyme is a glycoside hydrolase and the hydrolase cleavage site of each oligonucleotide detection reagent comprises a polysaccharide. In embodiments, the sample comprises at least two hydrolases, and wherein the hydrolases are a nuclease, a peptidase, a glycoside hydrolase or combinations thereof.

In embodiments, the disclosure provides an oligonucleotide detection reagent comprising: (i) a targeting agent complement: (ii) a targeting agent blocker that is complementary to at least a portion of the targeting agent complement; and (iii) a hydrolase cleavage site: wherein the targeting agent complement and the targeting agent blocker are hybridized.

In embodiments of the oligonucleotide detection reagent, the targeting agent complement and the targeting agent blocker are on a contiguous strand of the oligonucleotide detection reagent. In embodiments, the oligonucleotide detection reagent further comprises a detectable label.

In embodiments, the oligonucleotide detection reagent comprises, in 5′ to 3′ order, the targeting agent blocker, the hydrolase cleavage site, the targeting agent complement, and the detectable label. In embodiments, the targeting agent complement is on a first strand of the oligonucleotide detection reagent, and the targeting agent blocker and the hydrolase cleavage site are on a second strand of the oligonucleotide detection reagent.

In embodiments of the oligonucleotide detection reagent, the targeting agent blocker comprises a first region and a second region, wherein the hydrolase cleavage site is positioned between the first region and the second region; and wherein the first region and the second region of the targeting agent blocker hybridize to first and second regions of the targeting agent complement on the second strand. In embodiments, the targeting agent complement, the hydrolase cleavage site and the targeting agent blocker are comprised on a single strand.

In embodiments of the oligonucleotide detection reagent, the hydrolase cleavage site comprises a nucleic acid. In embodiments, the hydrolase cleavage site comprises a peptide. In embodiments, the hydrolase cleavage site comprises a polysaccharide.

In embodiments of the oligonucleotide detection reagent, the detectable label is detected by using light scattering, optical absorbance, fluorescence, chemiluminescence, electrochemiluminescence (ECL), bioluminescence, phosphorescence, radioactivity, magnetic field, or combinations thereof. In embodiments, the detectable label is an electrochemiluminescent label, and the detecting comprises measuring an ECL signal.

In embodiments, the disclosure provides a kit comprising: (a) at least one oligonucleotide detection reagent, wherein the oligonucleotide detection reagent comprises: (i) a targeting agent complement (TAC): (ii) a targeting agent blocker that is complementary to at least a portion of the TAC; and (ii) a hydrolase cleavage site; and (b) one or more detecting reagents for detecting an unblocked oligonucleotide.

In embodiments of the kit, the TAC and the targeting agent blocker are on a contiguous strand of the oligonucleotide detection reagent. In embodiments, the oligonucleotide detection reagent further comprises a detectable label. In embodiments, the oligonucleotide detection reagent comprises, in 5′ to 3′ order, the targeting agent blocker, the hydrolase cleavage site, the TAC, and the detectable label.

In embodiments of the kit, the TAC is on a first strand of the oligonucleotide detection reagent, and the targeting agent blocker and the hydrolase cleavage site are on a second strand of the oligonucleotide detection reagent. In embodiments, the targeting agent blocker comprises a first region and a second region, wherein the hydrolase cleavage site is positioned between the first region and the second region; and wherein the first region and the second region of the targeting agent blocker hybridize to first and second regions of the TAC on the second strand. In embodiments, the TAC, the hydrolase cleavage site and the targeting agent blocker are comprised on a single strand.

In embodiments of the kit, the hydrolase enzyme is a nuclease and the hydrolase cleavage site comprises a nucleic acid that is cleaved by the nuclease. In embodiments, the hydrolase enzyme is a peptidase and the hydrolase cleavage site comprises a peptide that is cleaved by the peptidase. In embodiments, the hydrolase enzyme is a glycoside hydrolase and the hydrolase cleavage site comprises a polysaccharide that is cleaved by the glycoside hydrolase.

In embodiments of the kit, the oligonucleotide detection reagent further comprises iv. a detectable label. In embodiments, the hydrolase cleavage site comprises a nucleic acid. In embodiments, the hydrolase cleavage site comprises a polypeptide. In embodiments, the hydrolase cleavage site comprises a polysaccharide.

In embodiments, the kit further comprises c) a detection surface comprising a targeting agent, wherein the targeting agent is a binding partner of the TAC, wherein uncleaved oligonucleotide detection reagent does not substantially bind to the detection surface.

In embodiments of the kit, the detecting reagents comprise a targeting agent, wherein the targeting agent is a binding partner of the TAC. In embodiments, the detecting reagents comprise one or more hybridization buffers. In embodiments, the detecting reagents comprise PCR extension buffers and free nucleotides.

In embodiments, the kit comprises at least two, three, four, five, six, seven, eight, nine, ten or more oligonucleotide detection reagents. In embodiments, each hydrolase cleavage site of the two or more oligonucleotide detection reagents comprises a nucleic acid, a peptide, a polysaccharide or combinations thereof.

In embodiments, the disclosure provides a method for detecting a hydrolase enzyme in a sample, comprising: a) contacting the sample with an oligonucleotide detection reagent, wherein the oligonucleotide detection reagent comprises: i) a primary targeting agent complement: ii) a secondary targeting agent complement: iii) a hydrolase cleavage site; and iv) a detectable label: wherein the hydrolase enzyme cleaves the oligonucleotide detection reagent, thereby generating (i) a cleaved secondary targeting agent complement and (ii) a first cleaved oligonucleotide detection reagent comprising the primary targeting agent complement and the detectable label: b) binding the cleaved secondary targeting agent complement, uncleaved oligonucleotide detection reagent, or both, to a binding surface comprising a secondary targeting agent that is a binding partner of the secondary targeting agent complement: c) immobilizing the first cleaved oligonucleotide detection reagent to a detection surface comprising a primary targeting agent, wherein the primary targeting agent is a binding partner of the primary targeting agent complement; and d) detecting the first cleaved oligonucleotide detection reagent immobilized on the detection surface, wherein the cleaved secondary targeting agent complement and the uncleaved oligonucleotide detection reagent on the binding surface are substantially undetected, thereby detecting the hydrolase enzyme in the sample.

In embodiments of the method, the secondary targeting agent complement is attached to a first terminus of the hydrolase cleavage site, a first terminus of the primary targeting agent complement is attached to a second terminus of the hydrolase cleavage site, and the detectable label is attached to a second terminus of the primary targeting agent complement.

In embodiments of the method, the hydrolase enzyme is a nuclease and the hydrolase cleavage site comprises a nucleic acid that is cleaved by the nuclease. In embodiments, the hydrolase enzyme is a peptidase and the hydrolase cleavage site comprises a peptide that is cleaved by the peptidase. In embodiments, the hydrolase enzyme is a glycoside hydrolase and the hydrolase cleavage site comprises a polysaccharide that is cleaved by the glycoside hydrolase.

In embodiments of the method, the primary targeting agent complement further comprises a nuclease-resistant nucleotide. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments of the method, the secondary targeting agent complement comprises biotin, and the secondary targeting agent comprises avidin or streptavidin. In embodiments, each of the primary targeting agent complement and the primary targeting agent is substantially unreactive with the secondary targeting agent and secondary targeting agent complement.

In embodiments, the method further comprises, prior to the detecting, separating the binding surface comprising the secondary targeting agent complement from the detection surface comprising the first cleaved oligonucleotide detection reagent.

In embodiments of the method, the primary targeting agent complement and the primary targeting agent comprise complementary oligonucleotides, and the immobilizing comprises hybridizing the primary targeting agent complement of the first cleaved oligonucleotide detection reagent to the primary targeting agent on the detection surface.

In embodiments of the method, the detectable label is detected by using light scattering, optical absorbance, fluorescence, chemiluminescence, electrochemiluminescence (ECL), bioluminescence, phosphorescence, radioactivity, magnetic field, or combinations thereof. In embodiments, the detectable label is an electrochemiluminescent label, and the detecting comprises measuring an ECL signal.

In embodiments, the detection surface comprises a particle. In embodiments, the detection surface comprises a well of a multi-well plate. In embodiments, the detection surface comprises an electrode. In embodiments, the binding surface is a particle.

In embodiments, the method further comprises, prior to step (c), separating the binding surface from the first cleaved oligonucleotide detection reagent.

In embodiments, the method is a multiplexed method for detecting multiple hydrolase enzymes in a sample, wherein step (a) comprises contacting the sample with a plurality of hydrolase enzymes and a plurality of oligonucleotide detection reagents, wherein each oligonucleotide detection reagent comprises a primary targeting agent complement, a secondary targeting agent complement, a hydrolase cleavage site, and a detectable label, wherein, each unique hydrolase enzyme cleaves an oligonucleotide detection reagent comprising a unique hydrolase cleavage site for the hydrolase enzyme to generate (I) a cleaved secondary targeting agent complement and (II) a first cleaved oligonucleotide detection reagent comprising a unique primary targeting agent complement, thereby generating (i) a plurality of secondary targeting agent complements and (ii) a plurality of first cleaved oligonucleotide detection reagents, wherein each first cleaved oligonucleotide detection reagent comprises a unique primary targeting agent complement: wherein step (b) comprises binding the plurality of secondary targeting agent complements, uncleaved oligonucleotide detection reagent, or both, to a binding surface comprising a plurality of secondary targeting agents: wherein step (c) comprises immobilizing the plurality of first cleaved oligonucleotide detection reagents to a detection surface comprising a plurality of primary targeting agents, wherein each primary targeting agent is a binding partner of a unique primary targeting agent complement; and wherein step (d) comprises detecting the plurality of first cleaved oligonucleotide detection reagents bound to the detection surface, wherein the plurality of secondary targeting agent complements and the uncleaved oligonucleotide detection reagent on the binding surface are substantially undetected, thereby detecting the multiple hydrolase enzymes in the sample.

In embodiments of the multiplexed method, each unique primary targeting agent complement comprises a unique oligonucleotide sequence.

In embodiments of the multiplex method, each hydrolase enzyme is a nuclease and the hydrolase cleavage site of each oligonucleotide detection reagent comprises a nucleic acid. In embodiments, each hydrolase enzyme is a peptidase and the hydrolase cleavage site of each oligonucleotide detection reagent comprises a peptide. In embodiments, each hydrolase enzyme is a glycoside hydrolase and the hydrolase cleavage site of each oligonucleotide detection reagent comprises a polysaccharide. In embodiments, the sample comprises at least two hydrolases, and wherein the hydrolases are a nuclease, a peptidase, a glycoside hydrolase or combinations thereof.

In embodiments, the disclosure provides an oligonucleotide detection reagent comprising: i) a primary targeting agent complement: ii) a secondary targeting agent complement: iii) a hydrolase cleavage site; and iv) a detectable label.

In embodiments of the oligonucleotide detection reagent, the secondary targeting agent complement is attached to a first terminus of the hydrolase cleavage site, a first terminus of the primary targeting agent complement is attached to a second terminus of the hydrolase cleavage site, and the detectable label is attached to a second terminus of the primary targeting agent complement.

In embodiments of the oligonucleotide detection reagent, the hydrolase cleavage site comprises a nucleic acid. In embodiments, the hydrolase cleavage site comprises a peptide. In embodiments, the hydrolase cleavage site comprises a polysaccharide.

In embodiments of the oligonucleotide detection reagent, the detectable label is detected by using light scattering, optical absorbance, fluorescence, chemiluminescence, electrochemiluminescence (ECL), bioluminescence, phosphorescence, radioactivity, magnetic field, or combinations thereof. In embodiments, the detectable label is an electrochemiluminescent label, and the detecting comprises measuring an ECL signal.

In embodiments, the disclosure provides a kit comprising: a) at least one oligonucleotide detection reagent, wherein the oligonucleotide detection reagent comprises: i) a primary targeting agent complement: ii) a secondary targeting agent complement: iii) a hydrolase cleavage site; and iv) a detectable label; and b) one or more detecting reagents for detecting a primary targeting agent complement.

In embodiments of the kit, the hydrolase cleavage site comprises a nucleic acid. In embodiments, the hydrolase cleavage site comprises a polypeptide. In embodiments, the hydrolase cleavage site comprises a polysaccharide.

In embodiments, the kit further comprises c) a detection surface comprising a primary targeting agent, wherein the primary targeting agent is a binding partner of the primary targeting agent complement, wherein uncleaved oligonucleotide detection reagent does not substantially bind to the detection surface.

In embodiments of the kit, the detecting reagents comprise a primary targeting agent, wherein the primary targeting agent is a binding partner of the primary targeting agent complement. In embodiments, the detecting reagents comprise a secondary targeting agent, wherein the secondary targeting agent is a binding partner of the secondary targeting agent complement. In embodiments, the secondary targeting agent complement is biotin and the secondary targeting agent is avidin or streptavidin. In embodiments, the detecting reagents comprise one or more hybridization buffers.

In embodiments, the kit comprises at least two, three, four, five, six, seven, eight, nine, ten or more oligonucleotide detection reagents. In embodiments, each hydrolase cleavage site of the two or more oligonucleotide detection reagents comprises a nucleic acid, a peptide, a polysaccharide or combinations thereof.

### BRIEF DESCRIPTION OF THE DRAWINGS

The following drawings form part of the present specification and are included to further demonstrate exemplary embodiments of certain aspects of the present invention.

FIG. 1 illustrates an embodiment of an oligonucleotide detection reagent described herein. An oligonucleotide detection reagent comprises, in 5′ to 3′ order, a targeting agent blocker, a hydrolase cleavage site and a targeting agent complement (TAC). The targeting agent blocker is hybridized to the TAC. In embodiments, the hydrolase cleavage is comprised in a loop structure between the TAC and targeting agent blocker as shown. In embodiments, the hydrolase cleavage site is capable of being cleaved by a hydrolase enzyme, as described herein.

FIG. 2 illustrates an embodiment of an oligonucleotide detection reagent comprising a detectable label described herein. An oligonucleotide detection reagent comprises, in 5′ to 3′ order, a targeting agent blocker, a hydrolase cleavage site, a targeting agent complement (TAC), and a detectable label. The targeting agent blocker is hybridized to the TAC. In embodiments, the hydrolase cleavage site is comprised in a loop structure between the TAC and targeting agent blocker as shown. In embodiments, the hydrolase cleavage site is capable of being cleaved by a hydrolase enzyme, as described herein.

FIG. 3 illustrates an embodiment of an oligonucleotide detection reagent described herein. An oligonucleotide detection reagent comprises first and second strands, wherein a TAC is on the first strand, and a targeting agent blocker and a hydrolase cleavage site are on the second strand. The targeting agent blocker comprises a first region and a second region, wherein the hydrolase cleavage site is positioned between the first region and the second region of the targeting agent blocker. The first and second regions of the targeting agent blocker hybridize to the first and second regions of the TAC. In embodiments, the hydrolase cleavage site is comprised in a loop structure between the targeting agent blocker first region and targeting agent blocker second region as shown. In embodiments, the hydrolase cleavage site is capable of being cleaved by a hydrolase, as described herein.

FIG. 4 illustrates an embodiment of an oligonucleotide detection reagent comprising a detectable label described herein. An oligonucleotide detection reagent comprises first and second strands, wherein a TAC and detectable label are on the first strand, and a targeting agent blocker and a hydrolase cleavage site are on the second strand. The targeting agent blocker comprises a first region and a second region, wherein the hydrolase cleavage site is positioned between the first region and the second region of the targeting agent blocker. The first and second regions of the targeting agent blocker hybridize to the first and second regions of the TAC. In embodiments, the hydrolase cleavage site is comprised in a loop structure between the targeting agent blocker 1^{st} region and targeting agent blocker 2^{nd} region as shown. In embodiments, the hydrolase cleavage site is capable of being cleaved by a hydrolase, as described herein.

FIG. 5 illustrates an embodiment of a method described herein. An oligonucleotide detection reagent comprises, in the order listed: a secondary targeting agent complement (TAC), a hydrolase cleavage site, a primary TAC, and a detectable label. A hydrolase enzyme cleaves the hydrolase cleavage site, thereby generating one or more of: (i) first cleaved oligonucleotide detection reagents, each comprising the primary TAC and detectable label; and (ii) cleaved secondary TACs. The reaction mixture sample is incubated on a binding surface comprising a secondary targeting agent to bind the one or more cleaved secondary TACs and uncleaved oligonucleotide detection reagents, thereby separating the cleaved secondary TACs and uncleaved oligonucleotide detection reagents from the first cleaved oligonucleotide detection reagent(s). The resulting reaction mixture sample is then incubated on a detection surface comprising a primary targeting agent to immobilize the one or more first cleaved oligonucleotide detection reagents onto the detection surface. In embodiments, the detectable label(s) of the immobilized first cleaved oligonucleotide detection reagent(s) are detected as described herein.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, a “loop structure” is a portion of the oligonucleotide detection reagent that is not hybridized with another part of the oligonucleotide detection reagent. In embodiments where the hydrolase cleavage site comprises a nucleic acid, the loop structure can be a hairpin loop, e.g., a non-hybridized region of nucleic acid between two hybridized nucleic acids. In embodiments where the hydrolase cleavage site comprises a peptide, the loop structure is formed by the presence of the peptide, and, in some embodiments, non-hybridizing nucleic acid on one or both ends of the peptide, in between two hybridized nucleic acids. In embodiments where the hydrolase cleavage site comprises a polysaccharide, the loop structure is formed by the presence of the polysaccharide, and, in some embodiments, non-hybridizing nucleic acid on one or both ends of the polysaccharide, in between two hybridized nucleic acids. In embodiments where the hydrolase cleavage site comprises a lipid, the loop structure is formed by the presence of the lipid, and, in some embodiments, non-hybridizing nucleic acid on one or both ends of the lipid, in between two hybridized nucleic acids.

As used herein. “complementary” in reference to an oligonucleotide means that the oligonucleotide or one or more regions thereof is capable of hydrogen bonding with a second oligonucleotide or one or more regions thereof. Complementary oligonucleotides and/or nucleic acids need not have complementarity at each nucleotide and may include one or more nucleotide mismatches, i.e., points at which hydrogen bonding does not occur. For example, complementary oligonucleotides can have at least 80%, at least 85%, at least 90%, at least 95%, at least 96%, at least 97%, at least 98%, or at least 99% of nucleotides hydrogen bond. By contrast. “fully complementary” or “100% complementary” in reference to oligonucleotides means that each nucleotide hydrogen bonds without any nucleotide mismatches.

The present invention provides highly sensitive and specific methods and kits for detecting hydrolase enzyme activity. The present methods advantageously utilize the cleavage activity of hydrolase enzymes to amplify the assay signal, thereby further increasing the sensitivity. The present methods can be used to detect nuclease, peptidase, glycoside hydrolase or lipase activity. Moreover, the present methods can be performed in a multiplexed format that can simultaneously detect multiple enzyme activities of interest, thereby reducing the sample volume requirement as well as time and resources otherwise required for performing multiple individual assays.

### Methods for Detecting Hydrolase Enzyme Activity Using Oligonucleotide Detection Reagents Comprising a Target Agent Blocker

In embodiments, the invention provides a method for detecting a hydrolase enzyme in a sample, comprising: (a) contacting the sample with an oligonucleotide detection reagent, wherein the oligonucleotide detection reagent comprises: (i) a targeting agent complement (TAC): (ii) a targeting agent blocker that is complementary to at least a portion of the TAC; and (iii) a hydrolase cleavage site: wherein the TAC and the targeting agent blocker are hybridized, wherein the hydrolase cleaves the oligonucleotide detection reagent at the hydrolase cleavage site, thereby (i) destabilizing hybridization of the TAC and the targeting agent blocker and (ii) generating an unblocked oligonucleotide comprising the TAC; and (b) detecting the unblocked oligonucleotide, thereby detecting the hydrolase enzyme in the sample.

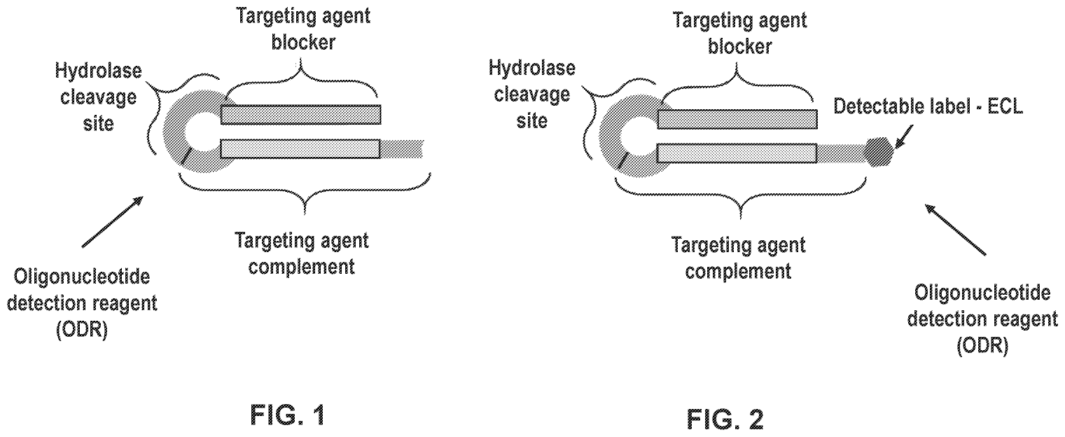

In embodiments of the oligonucleotide detection reagent, the TAC and the targeting agent blocker are on a contiguous strand of the oligonucleotide detection reagent. In embodiments, the TAC, the hydrolase cleavage site and the targeting agent blocker are comprised on a single strand. In embodiments, the TAC and the targeting agent blocker are on a contiguous strand of the oligonucleotide detection reagent as shown in the schematic of FIG. 1. In embodiments, the hydrolase cleavage is comprised in a loop structure between the TAC and targeting agent blocker as shown in FIG. 1.

In embodiments, the oligonucleotide detection reagent further comprises a detectable label that can be used for determining the presence of the TAC. In embodiments, the oligonucleotide detection reagent comprises, in 5′ to 3′ order: the targeting agent blocker, the hydrolase cleavage site, the TAC, and the detectable label. In embodiments of the oligonucleotide detection reagent, the targeting agent blocker, the hydrolase cleavage site, the TAC, and the detectable label are arranged in 5′ to 3′ order as shown in the schematic of FIG. 2. In embodiments, the hydrolase cleavage is comprised in a loop structure between the TAC and targeting agent blocker as shown in FIG. 2.

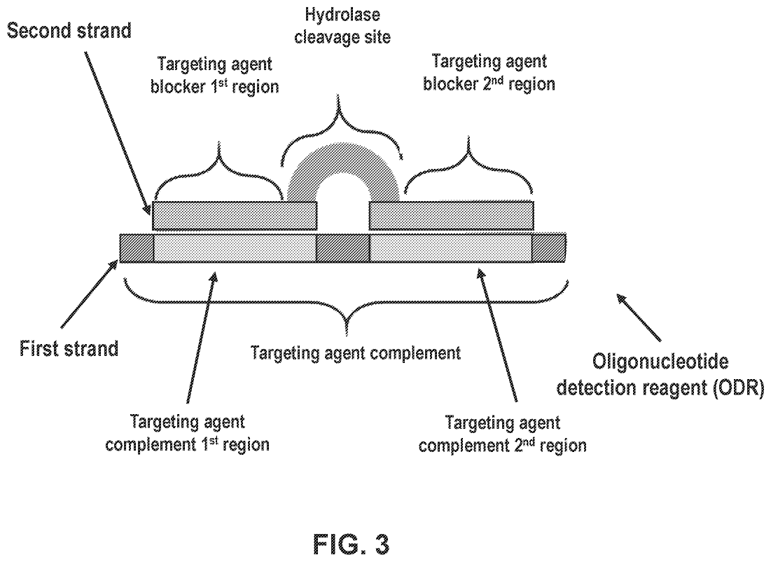

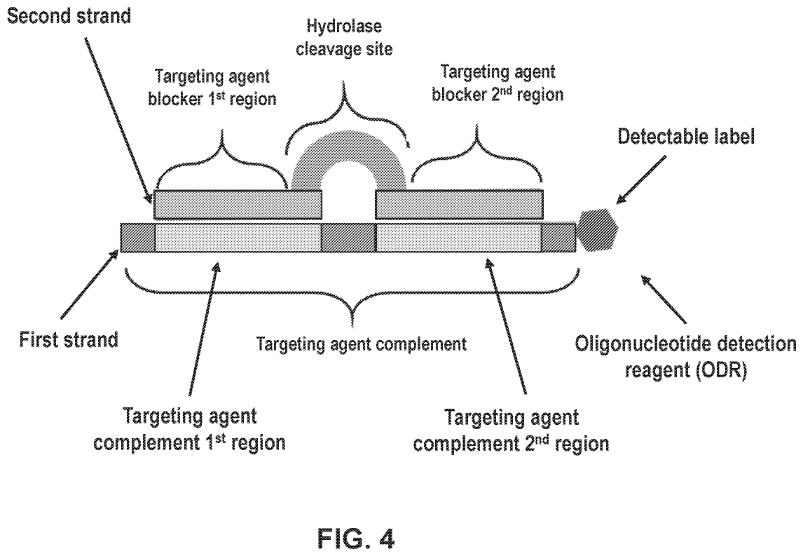

In embodiments of the oligonucleotide detection reagent, the TAC is on a first strand of the oligonucleotide detection reagent, and the targeting agent blocker and the hydrolase cleavage site are on a second strand of the oligonucleotide detection reagent. In embodiments where oligonucleotide detection reagent comprises a first and second strand, the targeting agent blocker comprises a first region and a second region, wherein the hydrolase cleavage site is positioned between the first region and the second region; and wherein the first region and the second region of the targeting agent blocker hybridize to first and second regions of the TAC on the second strand. In embodiments of the oligonucleotide detection reagent having a first strand and a second strand, the targeting agent blocker comprises a first region and a second region, wherein the hydrolase cleavage site is positioned between the first region and the second region; and wherein the first region and the second region of the targeting agent blocker hybridize to first and second regions of the TAC on the second strand as shown in FIG. 3. In embodiments, the hydrolase cleavage site is comprised in a loop structure between the targeting agent blocker 1^{st} region and targeting agent blocker 2^{nd} region as shown in FIG. 3. In embodiments having two strands and comprising a detectable label, the first stand comprises the TAC and a detectable label as shown in FIG. 4. In embodiments, the hydrolase cleavage site is comprised in a loop structure between the targeting agent blocker 1^{st} region and targeting agent blocker 2^{nd} region as shown in FIG. 4.

In embodiments, the oligonucleotide detection reagent is DNA. In embodiments, the oligonucleotide detection reagent is RNA. In embodiments, the oligonucleotide detection reagent is a combination of DNA and RNA. In embodiments, the oligonucleotide detection reagent is a nucleic acid with a non-natural backbone. As discussed in further detail herein, in embodiments, the hydrolase cleavage site in the oligonucleotide detection reagent can comprise a peptide, polysaccharide or lipid. In embodiments where the hydrolase cleavage site comprises a peptide, polysaccharide or lipid, the nucleic acid portions of the oligonucleotide detection reagent are DNA. In embodiments where the hydrolase cleavage site comprises a peptide, polysaccharide or lipid, the nucleic acid portions of the oligonucleotide detection reagent are RNA. In embodiments where the hydrolase cleavage site comprises a peptide, polysaccharide or lipid, the nucleic acid portions of the oligonucleotide detection reagent comprise a nucleic acid with a non-natural backbone. In embodiments, portions of the oligonucleotide detection reagent that are not part of the hydrolase cleavage site comprise nuclease resistant nucleotides. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments, the unblocked oligonucleotide is immobilized to a detection surface. In embodiments, the TAC is a binding partner of a targeting agent on a detection surface. In embodiments, hybridization of the TAC to the targeting agent blocker substantially prevents binding of the TAC to the targeting agent on the detection surface. As used herein, “substantially prevents binding” means that less than 20%, less than 15%, less than 10%, less than 5%, or less than 1% of the TAC that is hybridized to the targeting agent blocker, binds to the targeting agent on the detection surface. In embodiments, the TAC and the targeting agent comprise complementary oligonucleotides. In embodiments, the TAC and the targeting agent are fully complementary. In embodiments, the TAC and the targeting agent are at least 90%, at least 95%, at least 98%, at least 99%, or 100% complementary. In embodiments, the TAC of the unblocked oligonucleotide hybridizes to the targeting agent on the detection surface. In embodiments, uncleaved oligonucleotide detection reagent substantially does not bind to the detection surface, e.g., due to the targeting agent blocker substantially preventing binding of the TAC to the targeting agent as described herein. In embodiments, the cleaved targeting agent blocker does not bind to the detection surface.

In embodiments, the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length. In embodiments where the hydrolase cleavage site comprises a peptide, polysaccharide or lipid, the total length of the nucleic acid portions of the oligonucleotide detection reagent is about 20 to about 300, about 25 to about 280, about 30 to about 250), about 35 to about 220), about 40) to about 200, about 45 to about 180, about 50 to about 150, about 55 to about 120, about 60 to about 100, or about 65 to about 80 nucleotides in length.

In embodiments, the TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, or about 30 to about 35 nucleotides in length. In embodiments, the TAC and the targeting agent blocker are substantially the same length. In embodiments, the TAC and the targeting agent blocker differ in length by no more than 1, 2, 3, 4, 5, 6, 7, 8, 9, or 10 nucleotides. In embodiments, the targeting agent blocker is shorter than the TAC by about 1, 2, 3, 4, 5, 6, 7, 8, 9, or 10 nucleotides. In embodiments, the TAC is shorter than the targeting agent blocker by about 1, 2, 3, 4, 5, 6, 7, 8, 9, or 10 nucleotides. In embodiments, the difference in length between the targeting agent blocker and the TAC does not affect the targeting agent blocker's ability to substantially prevent binding of the TAC to the targeting agent on the detection surface. In embodiments, the TAC comprises any of SEQ ID NOs: 14-17.

### Methods for Detecting Hydrolase Enzyme Activity Using Oligonucleotide Detection Reagents Comprising a Secondary Targeting Agent

In embodiments, the invention provides a method for detecting a hydrolase enzyme in a sample, comprising: (a) contacting the sample with an oligonucleotide detection reagent, wherein the oligonucleotide detection reagent comprises: (i) a primary targeting agent complement (primary TAC): (ii) a secondary targeting agent complement (secondary TAC): (iii) a hydrolase cleavage site; and (iv) a detectable label: wherein the hydrolase enzyme cleaves the oligonucleotide detection reagent, thereby generating (i) a cleaved secondary TAC and (ii) a first cleaved oligonucleotide detection reagent comprising the primary TAC and the detectable label: (b) binding the cleaved secondary TAC, uncleaved oligonucleotide detection reagent, or both, to a binding surface comprising a secondary targeting agent that is a binding partner of the secondary TAC: (c) immobilizing the first cleaved oligonucleotide detection reagent to a detection surface comprising a primary targeting agent, wherein the primary targeting agent is a binding partner of the primary TAC; and (d) detecting the first cleaved oligonucleotide detection reagent bound to the detection surface, wherein the secondary TAC and the uncleaved oligonucleotide detection reagent on the binding surface are substantially undetected, thereby detecting the hydrolase enzyme in the sample.

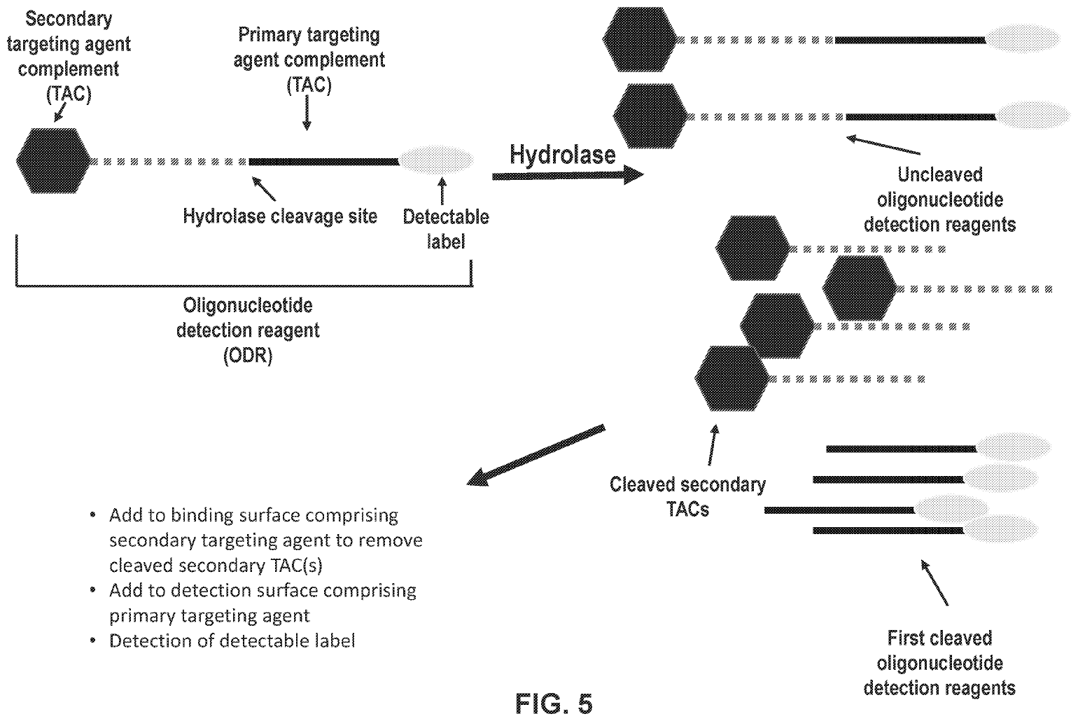

In embodiments, the oligonucleotide detection reagent comprises, in the order listed: a secondary TAC, a hydrolase cleavage site, a primary TAC, and a detectable label. In embodiments, a hydrolase enzyme cleaves the hydrolase cleavage site, thereby generating one or more of: (i) first cleaved oligonucleotide detection reagent, each comprising the primary TAC and detectable label; and (ii) cleaved secondary TACs, each comprising the secondary TAC. In embodiments, the secondary TAC, hydrolase cleavage site, primary TAC and detectable label are arranged as shown in FIG. 5.

In embodiments, the oligonucleotide detection reagent is DNA. In embodiments, the oligonucleotide detection reagent is RNA. In embodiments, the oligonucleotide detection reagent is a combination of DNA and RNA. In embodiments, the oligonucleotide detection reagent is a nucleic acid with a non-natural backbone. As discussed in further detail herein, in embodiments, the hydrolase cleavage site in the oligonucleotide detection reagent can comprise a peptide, polysaccharide or lipid. In embodiments where the hydrolase cleavage site comprises a peptide, polysaccharide or lipid, the nucleic acid portions of the oligonucleotide detection reagent are DNA. In embodiments where the hydrolase cleavage site comprises a peptide, polysaccharide or lipid, the nucleic acid portions of the oligonucleotide detection reagent are RNA. In embodiments where the hydrolase cleavage site comprises a peptide, polysaccharide or lipid, the nucleic acid portions of the oligonucleotide detection reagent comprise a nucleic acid with a non-natural backbone. In embodiments, portions of the oligonucleotide detection reagent that are not part of the hydrolase cleavage site comprise nuclease resistant nucleotides. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments, a reaction mixture comprising the first cleaved oligonucleotide detection reagent, cleaved secondary TAC, and uncleaved oligonucleotide detection reagent is formed following contacting the sample with the oligonucleotide detection reagent. In embodiments, the presence of uncleaved oligonucleotide detection reagent and/or cleaved secondary TAC interferes with one or more of the downstream steps of the method, e.g., immobilization and/or detection of the first cleaved oligonucleotide detection reagent. In embodiments, the cleaved secondary TAC and/or uncleaved oligonucleotide detection reagent are separated from the first cleaved oligonucleotide detection reagent. In embodiments, the separating comprises contacting the reaction mixture (comprising the first cleaved oligonucleotide detection reagent, cleaved secondary TAC, and uncleaved oligonucleotide detection reagent) with a binding surface comprising a secondary targeting agent, wherein the cleaved secondary TAC and the uncleaved oligonucleotide detection reagent bind to the binding surface. In embodiments, the first cleaved oligonucleotide detection reagent does not comprise the secondary TAC and therefore does not bind to the binding surface. In embodiments, the binding of the cleaved secondary TAC and the uncleaved oligonucleotide detection reagent to the binding surface reduces or eliminates interference with the immobilization and/or detection of the first cleaved oligonucleotide detection reagent.

In embodiments, the secondary TAC is a binding partner of the secondary targeting agent on the binding surface. In embodiments, the secondary TAC and the secondary targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the secondary targeting agent and secondary TAC are cross-reactive moieties, e.g., thiol and maleimide or iodoacetamide: aldehyde and hydrazide: or azide and alkyne or cycloalkyne. In embodiments, the secondary TAC comprises biotin, and the secondary targeting agent comprises avidin or streptavidin. In embodiments, the secondary TAC is an oligonucleotide of about 5 to about 100, about 6 to about 90, about 7 to about 80, about 8 to about 70, about 9 to about 60 nucleotides, about 10 to about 50), about 15 to about 45, about 20 to about 40, about 20 to about 30, or about 30 to about 35 nucleotides in length.

In embodiments, the method comprises, following binding of the cleaved secondary TAC and the uncleaved oligonucleotide detection reagent to the binding surface, immobilizing the first cleaved oligonucleotide detection reagent to a detection surface comprising a primary targeting agent.

In embodiments, the primary TAC is a binding partner of a primary targeting agent on a detection surface. In embodiments, the primary TAC and the primary targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the primary targeting agent and the primary TAC are cross-reactive moieties, e.g., thiol and maleimide or iodoacetamide: aldehyde and hydrazide: or azide and alkyne or cycloalkyne. In embodiments, the primary TAC and the primary targeting agent comprise complementary oligonucleotides. In embodiments, the primary TAC and the primary targeting agent are substantially unreactive with the secondary TAC and the secondary targeting agent. In embodiments, the primary TAC and the primary targeting agent comprise complementary oligonucleotides, and the secondary TAC and the secondary targeting agent comprise biotin and avidin/streptavidin. In embodiments, the primary TAC and the primary targeting agent comprise complementary oligonucleotides, and the secondary TAC and the secondary targeting agent comprise complementary oligonucleotides that are substantially non-hybridizable to the primary TAC or primary targeting agent. In embodiments, the primary TAC and the primary targeting agent comprise complementary oligonucleotides, and the immobilizing comprises hybridizing the primary TAC of the first cleaved oligonucleotide detection reagent to the primary targeting agent on the detection surface.

As used herein, the term “substantially unreactive” means that less than 10%, less than 5%, less than 2%, or less than 1% of either of the TAC or targeting agent reacts with either of the secondary TAC or secondary targeting agent. In embodiments, the TAC and the targeting agent comprise complementary oligonucleotides, and the secondary TAC and the secondary targeting agent comprise a non-oligonucleotide binding pair. In embodiments, the TAC and the targeting agent comprise complementary oligonucleotides, and the secondary TAC and the secondary targeting agent comprise complementary oligonucleotides that are substantially non-hybridizable to the TAC or targeting agent. As used herein, “substantially non-hybridizable” means that under standard nucleic acid hybridization conditions, less than 10%, less than 5%. less than 2%, or less than 1% of either of the TAC or targeting agent hybridizes with either of the secondary TAC or secondary targeting agent. Standard nucleic acid hybridization conditions are described, e.g., in Herzer and Englert, “Chapter 14. Nucleic Acid Hybridization,” in Molecular Biology Problem Solver: A Laboratory Guide, (2001) ed. Alan S. Gersterin: Wiley-Liss, Inc.

In embodiments, the primary TAC is an oligonucleotide of about 5 to about 100, about 6 to about 90, about 7 to about 80, about 8 to about 70, about 9 to about 60 nucleotides, about 10) to about 50), about 15 to about 45, about 20 to about 40, about 20 to about 30, about 20 to about 35, or about 30) to about 35 nucleotides in length. In embodiments, the primary TAC comprises any of SEQ ID NOs: 14-17.

In embodiments, the method comprises detecting the immobilized first cleaved oligonucleotide detection reagent on the detection surface. In embodiments, the cleaved secondary TAC and the uncleaved oligonucleotide detection reagent on the binding surface are substantially undetected. As used herein, the term “substantially undetected” means that less than 20%, less than 15%, less than 10%, less than 5%, less than 2%, or less than 1% of the total detected detectable label is from a detectable label that is not on the first cleaved oligonucleotide detection reagent. In embodiments, the binding surface prevents detection of the cleaved secondary TAC and the uncleaved oligonucleotide detection reagent. In embodiments, the method further comprises, prior to the detecting, separating the binding surface comprising the secondary TAC from the detection surface comprising the first cleaved oligonucleotide detection reagent. In embodiments, the separating comprises placing the binding surface at a distal location from the detection surface. In embodiments, the separating comprises placing the binding surface at a distance of at least about 10 μm from the detection surface, e.g., about 10, 15, 20, 25, 30, 35, 40, 45, 50, 60, 70, 80, 90, 100, or more than 100 μm from the detection surface. For example, in embodiments where the detectable label comprises an electrochemiluminescence (ECL) label and the detecting comprises applying a voltage waveform to the detection surface, generating an ECL signal, and detecting the ECL signal, the binding surface provides sufficient separation from the detection surface such that the uncleaved oligonucleotide detection reagent on the binding surface is substantially unresponsive to the voltage waveform and therefore does not generate a detectable ECL signal. In embodiments, the term “substantially unresponsive” means that less than 20%, less than 15%, less than 10%, less than 5%, less than 2%, or less than 1% of the total generated ECL signal is from an ECL label that is not on the first cleaved oligonucleotide detection reagent.

In embodiments, the detecting comprises measuring the amount of detectable label on the detection surface. In embodiments, the detectable label is detectable by light scattering, optical absorbance, fluorescence, chemiluminescence, electrochemiluminescence (ECL), bioluminescence, phosphorescence, radioactivity, magnetic field, or combinations thereof. In embodiments, the detectable label comprises phycoerythrin (PE). In embodiments, the detectable label comprises a β-galactosidase (β-gal) enzyme that can be detected by fluorescence detection when the β-gal enzyme cleaves a substrate such as resorufin-β-D-galactopyranoside to yield a fluorescent signal. In embodiments, the detectable label comprises an ECL label, and the detecting comprises measuring an ECL signal. In embodiments, the ECL label comprises ruthenium. ECL labels, ECL assays, and instrumentation for conducting ECL assays are further described herein.

In embodiments, the method comprises contacting the sample with multiple copies of the oligonucleotide detection reagent. In embodiments, the hydrolase enzyme cleaves the multiple copies of the oligonucleotide detection reagent, thereby generating a plurality of first cleaved oligonucleotide detection reagents.

In embodiments, the method comprises immobilizing the plurality of first cleaved oligonucleotide detection reagents to the detection surface; and detecting the plurality of immobilized first cleaved oligonucleotide detection reagents. In embodiments, the plurality of first cleaved oligonucleotide detection reagents amplifies the assay signal. In embodiments, the method has increased sensitivity for detecting the nucleic acid of interest as compared to a method that does not amplify the assay signal as described herein. In embodiments, the method is capable of detecting a lower amount of nucleic acid of interest in a sample as compared with a method that does not form the plurality of first cleaved oligonucleotide detection reagents as described herein.

### Oligonucleotide Detection Reagents Comprising a Targeting Agent Blocker

In embodiments, the invention provides an oligonucleotide detection reagent, comprising: (i) a targeting agent complement (TAC): (ii) a targeting agent blocker that is complementary to at least a portion of the TAC; and (iii) a hydrolase cleavage site: wherein the TAC and the targeting agent blocker are hybridized.

In embodiments of the oligonucleotide detection reagent, the TAC and the targeting agent blocker are on a contiguous strand of the oligonucleotide detection reagent. In embodiments, the TAC, the hydrolase cleavage site and the targeting agent blocker are comprised on a single strand. In embodiments, the TAC and the targeting agent blocker are on a contiguous strand of the oligonucleotide detection reagent as shown in the schematic of FIG. 1. In embodiments, the hydrolase cleavage is comprised in a loop structure between the TAC and targeting agent blocker as shown in FIG. 1.

In embodiments, the oligonucleotide detection reagent further comprises a detectable label that can be used for determining the presence of the TAC. In embodiments, the oligonucleotide detection reagent comprises, in 5′ to 3′ order: the targeting agent blocker, the hydrolase cleavage site, the TAC, and the detectable label. In embodiments of the oligonucleotide detection reagent, the targeting agent blocker, the hydrolase cleavage site, the TAC, and the detectable label are arranged in 5′ to 3′ order as shown in the schematic of FIG. 2. In embodiments, the hydrolase cleavage is comprised in a loop structure between the TAC and targeting agent blocker as shown in FIG. 2.

In embodiments of the oligonucleotide detection reagent, the TAC is on a first strand of the oligonucleotide detection reagent, and the targeting agent blocker and the hydrolase cleavage site are on a second strand of the oligonucleotide detection reagent. In embodiments where oligonucleotide detection reagent comprises a first and second strand, the targeting agent blocker comprises a first region and a second region, wherein the hydrolase cleavage site is positioned between the first region and the second region; and wherein the first region and the second region of the targeting agent blocker hybridize to first and second regions of the TAC on the second strand. In embodiments of the oligonucleotide detection reagent having a first strand and a second strand, the targeting agent blocker comprises a first region and a second region, wherein the hydrolase cleavage site is positioned between the first region and the second region; and wherein the first region and the second region of the targeting agent blocker hybridize to first and second regions of the TAC on the second strand as shown in FIG. 3. In embodiments, the hydrolase cleavage site is comprised in a loop structure between the targeting agent blocker 1^{st} region and targeting agent blocker 2^{nd} region as shown in FIG. 3. In embodiments having two strands and comprising a detectable label, the first stand comprises the TAC and a detectable label as shown in FIG. 4. In embodiments, the hydrolase cleavage site is comprised in a loop structure between the targeting agent blocker 1^{st} region and targeting agent blocker 2^{nd} region as shown in FIG. 4.

In embodiments, the oligonucleotide detection reagent is DNA. In embodiments, the oligonucleotide detection reagent is RNA. In embodiments, the oligonucleotide detection reagent is a combination of DNA and RNA. In embodiments, the oligonucleotide detection reagent is a nucleic acid with a non-natural backbone. As discussed in further detail herein, in embodiments, the hydrolase cleavage site in the oligonucleotide detection reagent can comprise a peptide, polysaccharide or lipid. In embodiments where the hydrolase cleavage site comprises a peptide, polysaccharide or lipid, the nucleic acid portions of the oligonucleotide detection reagent are DNA. In embodiments where the hydrolase cleavage site comprises a peptide, polysaccharide or lipid, the nucleic acid portions of the oligonucleotide detection reagent are RNA. In embodiments where the hydrolase cleavage site comprises a peptide, polysaccharide or lipid, the nucleic acid portions of the oligonucleotide detection reagent comprise a nucleic acid with a non-natural backbone. In embodiments, portions of the oligonucleotide detection reagent that are not part of the hydrolase cleavage site comprise nuclease resistant nucleotides. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments, the oligonucleotide detection reagent comprises more than one hydrolase cleavage site, e.g., 2, 3, 4, 5 or more hydrolase cleavage sites. In embodiments where the oligonucleotide detection reagent comprises more than one hydrolase cleavage site, each hydrolase cleavage site is cleaved by a different hydrolase.

In embodiments, the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length. In embodiments where the hydrolase cleavage site comprises a peptide, polysaccharide or lipid, the total length of the nucleic acid portions of the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length.

In embodiments, the TAC is an oligonucleotide of about 5 to about 100, about 6 to about 90, about 7 to about 80, about 8 to about 70, about 9 to about 60 nucleotides, about 10 to about 50), about 15 to about 45, about 20 to about 40, or about 30 to about 35 nucleotides in length. In embodiments, the TAC and the targeting agent blocker are substantially the same length. In embodiments, the TAC and the targeting agent blocker differ in length by no more than 1, 2, 3, 4, 5, 6, 7, 8, 9, or 10 nucleotides. In embodiments, the targeting agent blocker is shorter than the TAC by about 1, 2, 3, 4, 5, 6, 7, 8, 9, or 10 nucleotides. In embodiments, the TAC is shorter than the targeting agent blocker by about 1, 2, 3, 4, 5, 6, 7, 8, 9, or 10 nucleotides. In embodiments, the TAC comprises any of SEQ ID NOs: 14-17.

### Oligonucleotide Detection Reagents Comprising a Secondary Targeting Agent

In embodiments, the disclosure provides an oligonucleotide detection reagent comprising: i) a primary targeting agent complement: ii) a secondary targeting agent complement: iii) a hydrolase cleavage site; and iv) a detectable label.

In embodiments, the oligonucleotide detection reagent comprises, in the order listed: a secondary TAC, a hydrolase cleavage site, a primary TAC, and a detectable label. In embodiments, a hydrolase enzyme cleaves the hydrolase cleavage site, thereby generating one or more of: (i) first cleaved oligonucleotide detection reagent, each comprising the primary TAC and detectable label; and (ii) cleaved secondary TAC, each comprising the secondary TAC. In embodiments, the secondary TAC, hydrolase cleavage site, primary TAC and detectable label are arranged as shown in FIG. 5.

In embodiments of the oligonucleotide detection reagent, the secondary targeting agent complement is attached to a first terminus of the hydrolase cleavage site, a first terminus of the primary targeting agent complement is attached to a second terminus of the hydrolase cleavage site, and the detectable label is attached to a second terminus of the primary targeting agent complement.

In embodiments of the oligonucleotide detection reagent, the hydrolase cleavage site comprises a nucleic acid. In embodiments, the hydrolase cleavage site comprises a peptide. In embodiments, the hydrolase cleavage site comprises a polysaccharide.

In embodiments, the oligonucleotide detection reagent is DNA. In embodiments, the oligonucleotide detection reagent is RNA. In embodiments, the oligonucleotide detection reagent is a combination of DNA and RNA. In embodiments, the oligonucleotide detection reagent is a nucleic acid with a non-natural backbone. As discussed in further detail herein, in embodiments, the hydrolase cleavage site in the oligonucleotide detection reagent can comprise a peptide, polysaccharide or lipid. In embodiments where the hydrolase cleavage site comprises a peptide, polysaccharide or lipid, the nucleic acid portions of the oligonucleotide detection reagent are DNA. In embodiments where the hydrolase cleavage site comprises a peptide, polysaccharide or lipid, the nucleic acid portions of the oligonucleotide detection reagent are RNA. In embodiments where the hydrolase cleavage site comprises a peptide, polysaccharide or lipid, the nucleic acid portions of the oligonucleotide detection reagent comprise a nucleic acid with a non-natural backbone. In embodiments, portions of the oligonucleotide detection reagent that are not part of the hydrolase cleavage site comprise nuclease resistant nucleotides. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments, the oligonucleotide detection reagent comprises more than one hydrolase cleavage site, e.g., 2, 3, 4, 5 or more hydrolase cleavage sites. In embodiments where the oligonucleotide detection reagent comprises more than one hydrolase cleavage site, each hydrolase cleavage site is cleaved by a different hydrolase.

In embodiments where the hydrolase cleavage site comprises a peptide, the peptide can be oriented so that the nucleic acid portion of the oligonucleotide detection reagent is attached to the C terminus of the peptide. In embodiments where the hydrolase cleavage site comprises a peptide, the peptide can be oriented so that the nucleic acid portion of the oligonucleotide detection reagent is attached to the N terminus of the peptide. In embodiments, the nucleic acid portion of the oligonucleotide detection reagent is oriented so that the 5′ end of the nucleic acid is attached to the peptide. In embodiments, the nucleic acid portion of the oligonucleotide detection reagent is oriented so that the 3′ end of the nucleic acid is attached to the peptide.

In embodiments, the portions of the oligonucleotide detection reagent are arranged in one of the following orientations:
1. Secondary TAC-N-Peptide-C-5″-oligonucleotide-3′-detectable label
2. Secondary TAC-C-Peptide-N-5′-oligonucleotide-3′-detectable label
3. Secondary TAC-N-Peptide-C-3′-oligonucleotide-5″-detectable label
4. Secondary TAC-C-Peptide-N-3′-oligonucleotide-5′-detectable label

In embodiments, the portions of the oligonucleotide detection reagent are directly attached to one another. In embodiments, the portions of the oligonucleotide detection reagent are attached to one another through a linker. Linkers are described herein. In embodiments, the oligonucleotide detection reagent contains both direct attachments and attachments through linkers in a single oligonucleotide detection reagent.

In embodiments, the oligonucleotide detection reagent is about 20 to about 300, about 25 to about 280, about 30) to about 250, about 35 to about 220, about 40 to about 200, about 45 to about 180, about 50 to about 150), about 55 to about 120, about 60 to about 100, or about 65 to about 80 nucleotides in length. In embodiments where the hydrolase cleavage site comprises a peptide, polysaccharide or lipid, the total length of the nucleic acid portions of the oligonucleotide detection reagent is about 20 to about 300, about 25 to about 280), about 30 to about 250), about 35 to about 220, about 40 to about 200, about 45 to about 180, about 50 to about 150, about 55 to about 120, about 60 to about 100, or about 65 to about 80 nucleotides in length.

In embodiments, the primary TAC is a binding partner of a primary targeting agent on a detection surface. In embodiments, the primary TAC and the primary targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the primary targeting agent and the primary TAC are cross-reactive moieties, e.g., thiol and maleimide or iodoacetamide: aldehyde and hydrazide: or azide and alkyne or cycloalkyne. In embodiments, the primary TAC and the primary targeting agent comprise complementary oligonucleotides. In embodiments, the primary TAC and the primary targeting agent are substantially unreactive with the secondary TAC and the secondary targeting agent. In embodiments, the primary TAC and the primary targeting agent comprise complementary oligonucleotides, and the secondary TAC and the secondary targeting agent comprise biotin and avidin/streptavidin. In embodiments, the primary TAC and the primary targeting agent comprise complementary oligonucleotides, and the secondary TAC and the secondary targeting agent comprise complementary oligonucleotides that are substantially non-hybridizable to the primary TAC or primary targeting agent. In embodiments, the primary TAC and the primary targeting agent comprise complementary oligonucleotides, and the immobilizing comprises hybridizing the primary TAC of the first cleaved oligonucleotide detection reagent to the primary targeting agent on the detection surface.

In embodiments, the primary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, about 20 to about 35, or about 30 to about 35 nucleotides in length. In embodiments, the primary TAC comprises any of SEQ ID NOs: 14-17. In embodiments, the primary TAC and the primary targeting agent are substantially unreactive with the secondary TAC and the secondary targeting agent. Primary targeting agents, TACs, and detection surfaces are further described herein.

In embodiments, the secondary TAC is a binding partner of the secondary targeting agent on the binding surface. In embodiments, the secondary TAC and the secondary targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the secondary targeting agent and secondary TAC are cross-reactive moieties, e.g., thiol and maleimide or iodoacetamide: aldehyde and hydrazide: or azide and alkyne or cycloalkyne. In embodiments, the secondary TAC comprises biotin, and the secondary targeting agent comprises avidin or streptavidin. In embodiments, the secondary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, or about 30 to about 35 nucleotides in length.

### Hydrolase Enzyme Activities

Hydrolase enzymes are a class of enzymes that catalyze a chemical reaction that uses water to break a chemical bond. The methods and kits embodied herein can be used to detect any type of hydrolase enzyme activity that causes the cleavage of a single substrate molecule into two separate product molecules. In embodiments, the oligonucleotide detection reagent comprises a hydrolase cleavage site that is cleaved by a hydrolase enzyme to be detected.

In embodiments, the hydrolase enzyme is a nuclease, e.g., an enzyme that cleaves nucleic acids. In embodiments, the hydrolase enzyme is a peptidase or a protease, e.g., an enzyme that cleaves proteins, peptides or chains of amino acids. In embodiments, the hydrolase enzyme is a glycoside hydrolase, e.g., an enzyme that cleaves polysaccharides, e.g., polymeric carbohydrates composed of at least two monosaccharide units. In embodiments, the hydrolase enzyme is a lipase, e.g., an enzyme that cleaves the glycerol backbone of a lipid chain.

In embodiments, the hydrolase enzyme is a nuclease and the hydrolase cleavage site comprises a nucleic acid that is cleaved by the nuclease. In embodiments, the hydrolase enzyme is a peptidase and the hydrolase cleavage site comprises a peptide that is cleaved by the peptidase. In embodiments, the hydrolase enzyme is a glycoside hydrolase and the hydrolase cleavage site comprises a polysaccharide that is cleaved by the glycoside hydrolase. In embodiments, the hydrolase enzyme is a lipase and the hydrolase cleavage site comprises a lipid that is cleaved by the lipase.

In embodiments the hydrolase enzyme is a nuclease that is an endonuclease, e.g., a nuclease that cleaves a phosphodiester bond within a nucleic acid chain. In embodiments, the nuclease is a deoxyribonuclease. In embodiments, the nuclease is deoxyribonuclease I. In embodiments, the nuclease is a prokaryotic endonuclease selected from RecBCD endonuclease, T7 endonuclease, T4 endonuclease II, Bal 31 endonuclease, Endonuclease I, Micrococcal nuclease or Endonuclease II. In embodiments, the nuclease is a eukaryotic endonuclease selected from Neurospora endonuclease, SI nuclease, PI nuclease, Mung bean nuclease I, Ustilago nuclease, DNase I, AP endonuclease or Endo R.

In embodiments, the endonuclease is a restriction enzyme, e.g., an endonuclease that cleaves a nucleic acid within or near a specific recognition site. In embodiments, the nuclease is a Type I restriction enzyme. In embodiments, the nuclease is a Type II restriction enzyme. In embodiments, the nuclease is a Type III restriction enzyme. In embodiments, the nuclease is a Type IV restriction enzyme. In embodiments, the nuclease is a Type V restriction enzyme. In embodiments, the nuclease is an artificial restriction enzyme such as a zinc finger nuclease. In embodiments, the nuclease is a restriction enzyme selected from EcoRI, EcoRII, BamHI, HindIII, TaqI, NotI, HinFI, Sau3AI, PvuII, SmaI, HaeIII, HgaI, AluI, EcoRV, EcoP15I, KpnI, PstI, SacI, SalI, ScaI, SpeI, SphI, StuI or XbaI.

In embodiments, the hydrolase enzyme is a nuclease that cleaves RNA, e.g., an RNase. In embodiments the RNase is selected from RNase A, RNase T1 or RNase I.

In embodiments the hydrolase enzyme is a nuclease that cleaves DNA. In embodiments where the hydrolase enzyme is a nuclease, the nuclease cleaves RNA. In embodiments where the hydrolase enzyme is a nuclease, the nuclease cleaves both DNA and RNA. In embodiments where the hydrolase enzyme is a nuclease, the nuclease cleaves single-stranded DNA. In embodiments where the hydrolase enzyme is a nuclease, the nuclease cleaves single-stranded RNA. In embodiments where the hydrolase enzyme is a nuclease, the nuclease cleaves both single-stranded DNA and RNA.

In embodiments the hydrolase enzyme is a peptidase that is an endopeptidase, e.g., a peptidase that cleaves a peptide bond within a peptide chain. In embodiments, the peptidase is selected from trypsin, chymotrypsin, elastase, thermolysin, pepsin, glutamyl endopeptidase and neprilysin. In embodiments, the peptidase is a Cathepsin. In embodiments, the peptidase is selected from Cathepsin A, Cathepsin B, Cathepsin C, Cathepsin D, Cathepsin E, Cathepsin F, Cathepsin G, Cathepsin H, Cathepsin K, Cathepsin L1, Cathepsin L2, Cathepsin O, Cathepsin S, Cathepsin W and Cathepsin Z (or X). In embodiments, the peptidase is a Gelatinase. In embodiments, the peptidase is selected from Gelatinase A and Gelatinase B.

In embodiments the hydrolase enzyme is a peptidase that cleaves a peptide bond in a peptide, protein or other amino acid chain. In embodiments, the hydrolase enzyme is a protease. In embodiments the protease is selected from an HIV protease, proteinase 3 (also known as PRTN3), and renin. In embodiments, the protease is Serine protease PR3 (PRTN3). In embodiments, the protease is a Caspase, e.g., Caspase 1, Caspase 2, Caspase 3, Caspase 4, Caspase 5, Caspase 6, Caspase 7, Caspase 8, Caspase 9), Caspase 10, Caspase 11, Caspase 12, Caspase 13 or Caspase 14. In embodiments, the protease is a sequence specific clotting factor, e.g., Factor Xa, Factor VIa, Factor VIIIa, Factor IXa or Factor XIa.

In embodiments where the hydrolase enzyme is a glycoside hydrolase (also known as glycosidase), the glycoside hydrolase cleaves a glycosidic linkage within the chain of a polysaccharide. In embodiments where the hydrolase enzyme is a glycoside hydrolase, the glycoside hydrolase is the neuraminidase of influenza. In embodiments where the hydrolase enzyme is a glycoside hydrolase, the glycoside hydrolase is a lysozyme. In embodiments, where the hydrolase enzyme is a glycoside hydrolase, the glycoside hydrolase is an amylase. In embodiments where the hydrolase enzyme is a glycoside hydrolase, the glycoside hydrolase enzyme is selected from alpha-amylase, beta-amylase, gamma-amylase, cellulase, sucrase-isomaltase, mannosyl-oligosaccharide glucosidase, acid alpha-glucosidase, beta-glucosidase, lactase, glycogen debranching enzyme or pullulanase. In embodiments where the hydrolase enzyme is a glycoside hydrolase, the glycoside hydrolase enzyme is selected from hyaluronidase, beta-galactosidase, N-acetyl-beta-glucosaminidase, beta-acetylglucosaminidase, beta-glucuronidase, alpha-fucosidase or alpha-mannosidase.

In embodiments the hydrolase enzyme is a glycoside hydrolase that cleaves a glycosidic linkage is a polysaccharide having at least two monosaccharide units.

In embodiments where the hydrolase enzyme is a lipase, the lipase cleaves the glycerol backbone of a lipid fatty acid chain.

### Hydrolase Cleavage Site

In embodiments of the invention, the hydrolase cleavage site within the oligonucleotide detection reagent comprises a sequence that is recognized and cleaved by the hydrolase enzyme or enzymes to be detected. In embodiments, the hydrolase cleavage site is specific, and is cleaved by only one type of hydrolase. In embodiments, the hydrolase cleavage site is semispecific, and is cleaved by only a few types of hydrolases, e.g., two, three, four, five or more specific hydrolases. In embodiments, the hydrolase cleavage site is class specific, and is only cleaved by a class of hydrolases, e.g., a group of hydrolases having the same general substrate. As a non-limiting example, a substrate cleaved by the members of the Gelatinase family is a class specific substrate that could be used in making a class specific hydrolase cleavage site.

In embodiments, the oligonucleotide detection reagent comprises more than one hydrolase cleavage site, e.g., 2, 3, 4, 5 or more hydrolase cleavage sites. In embodiments where the oligonucleotide detection reagent comprises more than one hydrolase cleavage site, each hydrolase cleavage site is cleaved by a different hydrolase.

In embodiments the hydrolase cleavage site comprises a nucleic acid that comprises DNA, RNA or nucleic acid having a modified backbone. In embodiments, the hydrolase cleavage site comprises DNA. In embodiments, the hydrolase cleavage site comprises single-stranded DNA. In embodiments, the hydrolase cleavage site comprises RNA. In embodiments, the hydrolase cleavage site comprises single-stranded RNA. In embodiments, the hydrolase cleavage site comprises DNA and RNA. In embodiments, the hydrolase cleavage site comprises single-stranded DNA and RNA.

In embodiments where the hydrolase cleavage site is a nucleic acid, the oligonucleotide detection reagents can be constructed using nucleic acid synthesis methods known in the art. In embodiments, when the hydrolase cleavage site is a single stranded DNA or RNA sequence, this sequence can be synthesized as part of entirety of the oligonucleotide detection reagent. In embodiments, the nucleic acid of the hydrolase cleavage site can be synthesized separately from the other portions of the oligonucleotide detection reagent and then ligated to the portions to form the full oligonucleotide detection reagent. In embodiments, the hydrolase cleavage site can be RNA while the remaining portions of the oligonucleotide detection reagent are DNA. In embodiments, the hydrolase cleavage site can be DNA while the remaining portions of the oligonucleotide detection reagent are RNA.

In embodiments where the hydrolase cleavage site comprises a peptide, polysaccharide or lipid, the peptide, polysaccharide or lipid may be attached to the oligonucleotide portions of the oligonucleotide detection reagent through a direct chemical bond, i.e., a direct chemical bond between oligonucleotide and peptide, between oligonucleotide and polysaccharide or a between oligonucleotide and lipid. In other embodiments, a linker is used to attach the peptide, polysaccharide or lipid to the oligonucleotide. Linkers are described further herein.

In embodiments where the hydrolase cleavage site comprises a peptide, the peptide is linked to the nucleic acid portions of the oligonucleotide detection reagent using methods known in the art for forming bonds between nucleic acids and peptides or for linking nucleic acids and peptides using indirect means. In embodiments, oligonucleotide detection reagents having a hydrolase cleavage site comprising a peptide are formed using the method of Williams et al. (Peptidyl-Oligonucleotide Conjugates Demonstrate Efficient Cleavage of RNA in a Sequence Specific Manner, Bioconjugate Chem. 26:1129-1143 (2015)), which is hereby incorporated by reference herein. In embodiments, the peptide comprised in the hydrolase cleavage site is modified with a phosphate group attached to both its N-terminal and C-terminal. In embodiments, the phosphate attached to the N-terminal end of the peptide is reacted to become the 3′ phosphate of the 5′ portion of the oligonucleotide detection reagent (e.g., the portion comprising the targeting agent blocker) and the phosphate attached the C-terminal end of the is reacted to become the 5′ phosphate of the 3′ portion of the oligonucleotide detection reagent (e.g., the portion comprising TAC).

In embodiments, methods known in the art for developing peptide nucleic acid chimeras can be used to form oligonucleotide detection reagents having a hydrolase cleavage site comprising a peptide. Examples of such methods are in Debacker et al. (Next-Generation Peptide Nucleic Acid Chimeras Exhibit High Affinity and Potent Gene Silencing, Biochemistry 58(6):582-589 (2019)) and Chen et al. (Design of embedded chimeric peptide nucleic acids that efficiently enter and accurately reactivate gene expression in vivo, PNAS, 107(39): 16846-16851 (2010)), which are hereby incorporated by reference herein. In embodiments, the methods used in forming peptide nucleic acid backbones can be used to form a phosphate bound to a peptide bond that can be used to attached nucleic acids to peptides.

In embodiments, peptide nucleic acid conjugates can be formed using succinimidyl 4-[N-maleimidomethyl]cyclohexane-1-carboxylate (SMCC) or similar cross linking agents, such as cross linking agents sold by Thermo Fisher, Inc. In embodiments, for conjugation using a cross-linker, the peptide has a lysine at the C terminus and oligonucleotide has a 5′ thiol and 3″ amino group. The peptide is reacted with SMCC, to modify the lysine residue and introduce the thiol reactive maleimide group. In embodiments, the oligonucleotide is deprotected using DTT to release the 5′ thiol, followed by a desalting spin column to remove the DTT and deprotecting group.

In embodiments, the oligonucleotide portions of the oligonucleotide detection reagent are conjugated to a peptide through a linker. In embodiments, the linker is a substituted or unsubstituted C₂-C₂₀ alkyl, alkene or alkynyl chain.

In embodiments where the hydrolase cleavage site comprises a polysaccharide, the polysaccharide is linked to the nucleic acid portions of the oligonucleotide detection reagent using methods known in the art for forming bonds between nucleic acids and polysaccharides or for linking polysaccharides and nucleic acids using indirect means. In embodiments, the ends of the polysaccharide are modified with an amine group or a thiol group and a standard amine to thiol conjugative chemistry is used.

In embodiments, the oligonucleotide portions of the oligonucleotide detection reagent are conjugated to a polysaccharide through a linker. In embodiments, the linker is a substituted or unsubstituted C₂-C₂₀ alkyl, alkene or alkynyl chain.

In embodiments where the hydrolase cleavage site comprises a lipid, the lipid is linked to the nucleic acid portions of the oligonucleotide detection reagent using methods known in the art for forming bonds between nucleic acids and lipids or for linking lipids and nucleic acids using indirect means. In embodiments, the ends of the lipid are modified with an amine group or a thiol group and a standard amine to thiol conjugative chemistry is used. In embodiments, the nucleic acid is modified with a phosphoroamidite which is then reacted with a hydroxyl group on the lipid. In embodiments, the lipid is conjugated to a phosphate on the nucleic acid. In embodiments, the nucleic acid is coupled to the lipid by attaching avidin or streptavidin to the lipid and biotin to the nucleic acid. In embodiments, the lipid and nucleic acid are attached using a method disclosed in Beales et al., Advances in Colloid and Interface Sciences 207:290-305, 2014. In embodiments, the lipid and nucleic acid are attached using a method disclosed in Li et al., National Science Review: 7(12): 1933-9153, 2020. In embodiments, the lipid and nucleic acid are attached using a method disclosed in Winkler, Therapeutic Delivery 4(7):791-809, 2013.

In embodiments, the hydrolase cleavage site comprises a glycolipid and the glycolipid is conjugated to the nucleic acid portions of the oligonucleotide detection reagent using the polysaccharide portion of the glycolipid as discussed above.

In embodiments, the oligonucleotide portions of the oligonucleotide detection reagent are conjugated to a polysaccharide through a linker. In embodiments, the linker is a substituted or unsubstituted C₂-C₂₀ alkyl, alkene or alkynyl chain.

### Detection of the Targeting Agent Complement

In embodiments, when the targeting agent complement (TAC) is unblocked due to cleavage the hydrolase cleavage site, the TAC is detected to determine that hydrolase activity has occurred. In embodiments, the TAC is detected by hybridizing the TAC to a targeting agent. In embodiments, the targeting agent is immobilized on a detection surface. In embodiments, the TAC comprises a detectable label. In embodiments, the targeting agent is a nucleic acid that is amplified using the TAC as a primer.

In embodiments of the method herein, the method further comprises, prior to detecting the unblocked oligonucleotide, immobilizing the unblocked oligonucleotide to a detection surface comprising a targeting agent, wherein the targeting agent is a binding partner of the TAC, wherein uncleaved oligonucleotide detection reagent does not substantially bind to the detection surface. In embodiments, detecting the unblocked oligonucleotide comprises detecting if the unblocked oligonucleotide is bound to the detection surface.

In embodiments, the unblocked oligonucleotide is immobilized to the detection surface. In embodiments, the TAC is a binding partner of a targeting agent on a detection surface. In embodiments, hybridization of the TAC to the targeting agent blocker substantially prevents binding of the TAC to the targeting agent on the detection surface. As used herein, “substantially prevents binding” means that less than 20%, less than 15%, less than 10%, less than 5%, or less than 1% of the TAC that is hybridized to the targeting agent blocker, binds to the targeting agent on the detection surface. In embodiments, the TAC and the targeting agent comprise complementary oligonucleotides. In embodiments, the TAC and the targeting agent are fully complementary. In embodiments, the TAC and the targeting agent are at least 90%, at least 95%, at least 98%, at least 99%, or 100% complementary. In embodiments, the TAC of the unblocked oligonucleotide hybridizes to the targeting agent on the detection surface. In embodiments, uncleaved oligonucleotide detection reagent substantially does not bind to the detection surface, e.g., due to the targeting agent blocker substantially preventing binding of the TAC to the targeting agent as described herein. In embodiments, the cleaved targeting agent blocker does not bind to the detection surface.

In embodiments, the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length. In embodiments where the hydrolase cleavage site comprises a peptide, polysaccharide or lipid, the total length of the nucleic acid portions of the oligonucleotide detection reagent is about 20 to about 300, about 25 to about 280, about 30 to about 250), about 35 to about 220, about 40 to about 200, about 45 to about 180, about 50 to about 150, about 55 to about 120, about 60) to about 100, or about 65 to about 80 nucleotides in length. In embodiments, the TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, or about 30 to about 35 nucleotides in length. In embodiments, the TAC and the targeting agent blocker are substantially the same length. In embodiments, the TAC and the targeting agent blocker differ in length by no more than 1, 2, 3, 4, 5, 6, 7, 8, 9, or 10 nucleotides. In embodiments, the targeting agent blocker is shorter than the TAC by about 1, 2, 3, 4, 5, 6, 7, 8, 9, or 10 nucleotides. In embodiments, the TAC is shorter than the targeting agent blocker by about 1, 2, 3, 4, 5, 6, 7, 8, 9, or 10 nucleotides. In embodiments, the difference in length between the targeting agent blocker and the TAC does not affect the targeting agent blocker's ability to substantially prevent binding of the TAC to the targeting agent on the detection surface. In embodiments, the TAC comprises any of SEQ ID NOs: 14-17.

In embodiments, the method comprises detecting the immobilized unblocked oligonucleotide immobilized on the detection surface. In embodiments, the components that are not bound to the detection surface, e.g., uncleaved oligonucleotide detection reagent and/or cleaved targeting agent blocker, are removed from the reaction mixture, e.g., by washing, prior to the detecting step. In embodiments, the uncleaved oligonucleotide detection reagent and the cleaved targeting agent blocker are substantially undetected.

In embodiments, the TAC and the targeting agent comprise complementary oligonucleotides, and the immobilizing comprises hybridizing the TAC of the unblocked oligonucleotide to the targeting agent on the detection surface.

In embodiments of the method, detecting the unblocked oligonucleotide comprises: i) using at least a portion of the unblocked oligonucleotide as a primer in an extension reaction to form an extension product; and ii) detecting the extension product.

In embodiments, the unblocked oligonucleotide comprises a primer for polymerase chain reaction (PCR), ligase chain reaction (LCR), strand displacement amplification (SDA), self-sustained synthetic reaction (3SR), or an isothermal amplification method. In embodiments, the unblocked oligonucleotide comprises a primer for an isothermal amplification method. In embodiments, the isothermal amplification method is helicase-dependent amplification. In embodiments, the isothermal amplification method is rolling circle amplification (RCA). Amplification methods are further described, e.g., in Carrino et al., J Microbiol Method 23(1):3-20 (1995): Fakruddin et al., J Pharm Bioallied Sci 5(4):245-252 (2013); and Nolte et al., “Chapter 1: Nucleic Acid Amplification Methods Overview” in Molecular Microbiology: Diagnostic Principles and Practice, 3^{rd} Ed. (2016), ASM Press. RCA is further described, e.g., in Baner et al., Nucleic Acids Res, 26:5073-5078 (1998); Lizardi et al., Nature Genetics 19:226 (1998): Schweitzer et al., Proc Natl Acad Sci USA 97:10113-10119 (2000); Faruqi et al., BMC Genomics 2:4 (2000): Nallur et al., Nucleic Acids Res 29:e118 (2001); Dean et al. Genome Res 11:1095-1099 (2001): Schweitzer et al., Nature Biotechnol 20:359-365 (2002); and U.S. Pat. Nos. 6,054,274; 6,291,187; 6,323,009; 6,344,329; and 6,368,801. In embodiments, the unblocked oligonucleotide primer is about 10 to about 50, about 11 to about 45, about 12 to about 40), about 13 to about 35, about 14 to about 30), about 15 to about 40, about 20 to about 35, about 25 to about 30), or about 30) to about 35 nucleotides in length. In embodiments, the primer is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In embodiments, the extension product is detected by hybridizing the extension product to a complementary sequence comprising a detectable label. In embodiments, the hybridized extension product and complementary sequence comprising a detectable label are purified using, for example, a size separation method before detection. In embodiments, purification using a size separation method is performed to remove non-extended nucleic acid, uncleaved oligonucleotide detection reagent and/or other nucleic acids that are present in the sample. In embodiments, the size separation is by gel electrophoresis. In embodiments, the size separation is by size exclusion chromatography. In embodiments, the size separation is by gel filtration chromatography. In embodiments, the extension product is detected by hybridization to an array. In embodiments, the array is a solid-phase array. In embodiments, the array is a bead array.

In embodiments where the oligonucleotide detection reagent comprises a detectable label, the detecting comprises measuring the amount of detectable label on the detection surface. In embodiments, the detectable label is detectable by light scattering, optical absorbance, fluorescence, chemiluminescence, electrochemiluminescence (ECL), bioluminescence, phosphorescence, radioactivity, magnetic field, or combinations thereof. In embodiments, the detectable label comprises a fluorescent label. In embodiments the detectable label comprises phycoerythrin (PE). In embodiments, the detectable label comprises a β-galactosidase (β-gal) enzyme that can be detected by fluorescence detection when the β-gal enzyme cleaves a substrate such as resorufin-β-D-galactopyranoside to yield a fluorescent signal. In embodiments, the detectable label comprises an ECL label, and the detecting comprises measuring an ECL signal. In embodiments, the ECL label comprises ruthenium. ECL labels, ECL assays, and instrumentation for conducting ECL assays are further described herein.

In embodiments, the detection surface comprises a particle. In embodiments, the detection surface comprises a magnetic bead or a magnetic microparticle. In embodiments, the detection surface comprises a microsphere internally dyed with varying proportions of red and infrared fluorophores that correspond to a distinct spectral signature, or bead region. In embodiments, the detection surface comprises a well of a multi-well plate. In embodiments, the detection surface comprises an electrode.

In embodiments where the oligonucleotide detection reagent comprises a detectable label, the method comprises immobilizing the plurality of unblocked oligonucleotides to the detection surface; and detecting the plurality of immobilized unblocked oligonucleotides. In embodiments, the plurality of unblocked oligonucleotides amplifies the assay signal. In embodiments, the method has increased sensitivity for detecting the nucleic acid of interest as compared to a method that does not amplify the assay signal as described herein. In embodiments, the method is capable of detecting a lower amount of nucleic acid of interest in a sample as compared with a method that does not form the plurality of unblocked oligonucleotides as described herein.

Multiplexed Embodiments for Oligonucleotide Detection Reagents that Comprise a Targeting Agent Blocker

In embodiments, the method is a multiplexed method for detecting multiple hydrolase enzyme activities of interest in a sample using oligonucleotide detection reagents that comprise a targeting agent blocker. In embodiments, the multiplexed method detects multiple hydrolase enzyme activities of interest simultaneously or substantially simultaneously.

In embodiments, the multiplexed method comprises: (a) contacting the sample with a plurality of oligonucleotide detection reagents, wherein each oligonucleotide detection reagent comprises a targeting agent complement (TAC), a targeting agent blocker hybridized to the TAC, a hydrolase cleavage site, and a detectable label, wherein, for each hydrolase enzyme in the sample, a hydrolase cleaves an oligonucleotide detection reagent comprising a unique hydrolase cleavage site for the hydrolase, thereby generating a plurality of unblocked oligonucleotides, wherein each unblocked oligonucleotide comprises a unique TAC: wherein, prior to detecting step (b), the plurality of unblocked oligonucleotides are immobilized to a detection surface comprising a plurality of targeting agents, wherein each targeting agent is a binding partner of a unique TAC, and wherein uncleaved oligonucleotide detection reagent does not substantially bind to the detection surface; and (b) detecting the plurality of unblocked oligonucleotides bound to the detection surface, thereby detecting the one or more hydrolase enzymes in in the sample.

In embodiments, an oligonucleotide detection reagent that corresponds to a unique hydrolase enzyme activity of interest comprises a unique TAC. In embodiments, each primary TAC comprises a unique oligonucleotide sequence that is substantially non-hybridizable to any other unique oligonucleotide sequence in the plurality of oligonucleotide detection reagents. In embodiments, the detection surface comprises multiple binding domains, wherein each binding domain comprises a unique targeting agent. Thus, in embodiments, the unblocked oligonucleotide, comprising a unique primary TAC, immobilized in each binding domain corresponds to a unique nucleic acid of interest. Binding domains are further described herein.

In embodiments of the multiplexed method, each hydrolase enzyme is a nuclease and the hydrolase cleavage site of each oligonucleotide detection reagent comprises a nucleic acid. In embodiments, each hydrolase enzyme is a peptidase and the hydrolase cleavage site of each oligonucleotide detection reagent comprises a peptide. In embodiments, each hydrolase enzyme is a glycoside hydrolase and the hydrolase cleavage site of each oligonucleotide detection reagent comprises a polysaccharide. In embodiments, each hydrolase enzyme is a lipase and the hydrolase cleavage site of each oligonucleotide detection reagent comprises a lipid.

In embodiments of the multiplexed method, the sample comprises at least two unique hydrolase enzymes, and wherein the hydrolases are a nuclease, a peptidase, a glycoside hydrolase, a lipase or combinations thereof. In embodiments, the sample comprises at least two unique nucleases. In embodiments, the sample comprises at least two unique peptidases. In embodiments, the sample comprises at least two unique glycoside hydrolases. In embodiments, the sample comprises at least two unique lipases. In embodiments, the sample comprises at least 2, 3, 4, 5, 6, 7, 8, 9, 10, 11, 12, 13, 14, 15, 16, 17, 18, 19 or 20 unique hydrolase enzymes. In embodiments, the multiplexed assays detect more than one type of hydrolase enzyme activity, e.g., combinations of nuclease, peptide, glycoside hydrolase and lipase activities. In embodiments, the multiplexed assays detect only one type of hydrolase activity, e.g., only nuclease, only peptidase only glycoside hydrolase or only lipase activity.

In embodiments, the multiple hydrolase enzyme activities of interest are detected by detecting the unblocked oligonucleotide in the binding domains, wherein each binding domain corresponds to a unique nucleic acid of interest. In embodiments, each of the plurality of oligonucleotide detection reagents comprises a same detectable label, and the unique nucleic acids of interest are detected based on the unblocked oligonucleotides immobilized in their corresponding binding domains. In embodiments, each unique oligonucleotide detection reagent comprises a unique detectable label, and the unique hydrolase enzyme activities of interest are detected based on the unique detectable labels. Detectable labels and detection methods are further described herein. In embodiments, a portion of the unique TAC from each unique oligonucleotide detection reagent can be used as a primer that can be extended to form a unique extended oligonucleotide, and the unique hydrolase enzyme activities of interest are detected based on the unique extended oligonucleotides. Extended oligonucleotides and their detection are further described herein.

An exemplary protocol for performing the multiplexed method comprises:

1A. Preparing the samples comprising the hydrolase enzyme activity or activities of interest. In embodiments, the preparing comprises obtaining the sample and extracting or purifying the hydrolase enzyme. In embodiments, the preparing does not require any extracting or purifying of the sample. In embodiments, the preparing comprises diluting the sample with a suitable buffer. In embodiments, the preparing does not require diluting the sample.

1B. Preparing the oligonucleotide detection reagent. In embodiments where the TAC and the targeting agent blocker are on first and second oligonucleotide strands, the preparing comprises mixing the first strand comprising the TAC and an excess of the second strand comprising the targeting agent blocker, heating the mixture to about 90° C., to about 98° C. (e.g., about 95° C.), and cooling the mixture by about 1° C., per minute to about 20° C., to allow the first and second strands to hybridize.

2A. Incubating sample reaction mixture(s), comprising the oligonucleotide detection reagents (e.g., about 0.05 nM to about 100 nM, about 0.1 nM to about 80 nM, about 0.2 nM to about 60 nM, about 0.3 nM to about 50 nM, about 0.4 nM to about 40 nM, about 0.1 nM to about 20 nM, or about 0.1 nM to about 10 nM of one or more oligonucleotide detection reagents, each one comprising a unique TAC), and the samples that comprise the multiple hydrolase enzyme activities of interest. In embodiments, the sample reaction mixture is in an assay buffer of pH about 6 to about 8, about 6.5 to about 7.5, or about 6.7 to about 7. In embodiments, the sample reaction mixture comprises a reaction volume of about 10 μL to about 1 mL, about 20 μL to about 700 μL, about 50 μL to about 500 μL, about 70 μL to about 200 μL, about 90 to about 150 μL, or about 100 μL. In embodiments, the sample reaction mixture is incubated for about 10 minutes to about 6 hours, about 30 minutes to about 4 hours, or about 1 hour to about 3 hours. In embodiments, the sample reaction mixture is incubated at about 20° C. to about 50° C., about 25° C., to about 45° C., about 30° C., to about 40° C., or about 37° C. In embodiments, the sample reaction mixture is incubated for about 1 hour to about 3 hours at about 37° C.

2B. Preparing an assay plate. In embodiments, the assay plate comprises multiple binding domains in each well, wherein each binding domain comprises a unique primary targeting agent that corresponds to a unique TAC on the oligonucleotide detection reagent. In embodiments, the assay plate is washed, e.g., with PBS, prior to Step 3 below. In embodiments, following the washing, a hybridization buffer is added to the assay plate (e.g., about 10 μL to about 50 μL, about 20 μL to about 40 μL, or about 30 μL per well of the assay plate). In embodiments, the hybridization buffer facilitates binding of the TAC to the targeting agent.

In embodiments, steps 1 and 2 are performed simultaneously or substantially simultaneously.

3A. Incubating the sample reaction on the assay plate comprising the hybridization buffer. In embodiments, about 10 μL to about 100 μL, about 20 μL to about 80 μL, about 30 μL to about 70 μL, about 40 μL to about 60 μL, or about 50 μL of the sample reaction is added to a well of the assay plate. In embodiments, the sample reaction is incubated for about 10 minutes to about 4 hours, about 30) minutes to about 2 hours, or about 1 hour. In embodiments, the sample reaction is incubated at about 15° C., to about 40° C., about 20° C., to about 37° C., about 25° C., to about 30° C., or about 27° C. In embodiments, the sample reaction is incubated for about 1 hour at about 27° C. In embodiments, the assay plate is washed, e.g., with PBS, following the incubating. In embodiments, when the assay plate is washed the remaining portions of cleaved oligonucleotide and/or uncleaved oligonucleotide detection reagent are removed.

Optional step 3B. Removing the remaining portions of cleaved oligonucleotide and/or uncleaved oligonucleotide detection reagent. In embodiments, the removing comprises contacting the sample reaction with magnetic beads comprising a secondary sequence complementary to a portion of the oligonucleotide detection reagent that is not part of the TAC. In embodiments, the secondary sequence is a binding partner of an amplification blocker on the oligonucleotide detection reagent. In embodiments, the magnetic beads are incubated with the sample reactions for about 10 minutes to about 4 hours, about 30 minutes to about 2 hours, or about 1 hour. In embodiments, the magnetic beads are incubated with the sample reaction at about 20° C., to about 50° C., about 25° C., to about 45° C., about 30° C., to about 40° C., or about 37° C. In embodiments, the magnetic beads are incubated with the sample reaction for about 1 hour at about 37° C. In embodiments, following the incubation, the beads are removed or separated (e.g., concentrated on a side of the sample reaction container such that the beads are no longer in contact with the sample reaction) from the sample reaction by contacting the sample reaction container with a magnet.

4. Reading the plate. In embodiments, about 50 μL to about 500 μL, about 100 μL to about 300 μL, or about 150 μL of a read buffer is added to the assay plate well. In embodiments, the assay plate is read on a plate reader immediately or substantially immediately following addition of the read buffer.

### Multiplexed Embodiments for Oligonucleotide Detection Reagents Comprising a Secondary Targeting Agent

In embodiments, the method is a multiplexed method for detecting multiple hydrolase enzymes in a sample using oligonucleotide detection reagents that lack a targeting agent blocker. In embodiments, each oligonucleotide detection reagent comprises a unique hydrolase cleavage site. In embodiments, the multiplexed method detects multiple hydrolase enzymes simultaneously or substantially simultaneously.

In embodiments, the multiplexed method comprises: (a) contacting the sample with a plurality of oligonucleotide detection reagents, wherein each oligonucleotide detection reagent comprises a primary TAC, a secondary TAC, a unique hydrolase cleavage site and a detectable label, wherein, each unique hydrolase enzyme cleaves an oligonucleotide detection reagent comprising a unique hydrolase cleavage site to generate (1) a cleaved secondary TAC and (2) a first cleaved oligonucleotide detection reagent comprising a unique primary TAC, thereby generating (i) a plurality of secondary TACs and (ii) a plurality of first cleaved oligonucleotide detection reagents, wherein each first cleaved oligonucleotide detection reagent comprises a unique primary TAC: (b) binding the plurality of secondary TACs, uncleaved oligonucleotide detection reagent, or both, to a binding surface comprising a plurality of secondary targeting agents: (c) immobilizing the plurality of first cleaved oligonucleotide detection reagents to a detection surface comprising a plurality of primary targeting agents, wherein each primary targeting agent is a binding partner of a unique primary TAC; and (d) detecting the plurality of first cleaved oligonucleotide detection reagents bound to the detection surface, wherein the plurality of secondary TACs and the uncleaved oligonucleotide detection reagent on the binding surface are substantially undetected, thereby detecting the multiple hydrolase enzymes in the sample.

In embodiments, each of the plurality of oligonucleotide detection reagents comprises a same secondary TAC. In embodiments, the secondary TAC of each oligonucleotide detection reagent comprises biotin. Thus, in embodiments, the binding surface is capable of binding to all of the cleaved secondary TACs and uncleaved oligonucleotide detection reagents. In embodiments, the binding of the cleaved secondary TACs and uncleaved oligonucleotide detection reagents to the binding surface reduces or eliminates interference with immobilization and/or detection of the plurality of first cleaved oligonucleotide detection reagents, as described herein.

In embodiments of the multiplexed method, each hydrolase enzyme is a nuclease and the hydrolase cleavage site of each oligonucleotide detection reagent comprises a nucleic acid. In embodiments, each hydrolase enzyme is a peptidase and the hydrolase cleavage site of each oligonucleotide detection reagent comprises a peptide. In embodiments, each hydrolase enzyme is a glycoside hydrolase and the hydrolase cleavage site of each oligonucleotide detection reagent comprises a polysaccharide. In embodiments, each hydrolase enzyme is a lipase and the hydrolase cleavage site of each oligonucleotide detection reagent comprises a lipid.

In embodiments of the multiplexed method, the sample comprises at least two unique hydrolase enzymes, and wherein the hydrolases are a nuclease, a peptidase, a glycoside hydrolase, a lipase or combinations thereof. In embodiments, the sample comprises at least two unique nucleases. In embodiments, the sample comprises at least two unique peptidases. In embodiments, the sample comprises at least two unique glycoside hydrolases. In embodiments, the sample comprises at least two unique lipases. In embodiments, the sample comprises at least 2, 3, 4, 5, 6, 7, 8, 9, 10, 11, 12, 13, 14, 15, 16, 17, 18, 19 or 20 unique hydrolase enzymes. In embodiments, the multiplexed assays detect more than one type of hydrolase enzyme activity, e.g., combinations of nuclease, peptide, glycoside hydrolase and lipase activities. In embodiments, the multiplexed assays detect only one type of hydrolase activity, e.g., only nuclease, only peptidase only glycoside hydrolase or only lipase activity.

In embodiments, an oligonucleotide detection reagent that corresponds to a unique nucleic acid of interest comprises a unique primary TAC. In embodiments, each unique primary TAC comprises a unique oligonucleotide sequence that is substantially non-hybridizable to any other unique oligonucleotide sequence in the plurality of oligonucleotide detection reagents. In embodiments, the detection surface comprises multiple binding domains, wherein each binding domain comprises a unique primary targeting agent. Thus, in embodiments, the first cleaved oligonucleotide detection reagent, comprising a unique primary TAC, immobilized in each binding domain corresponds to a unique nucleic acid of interest. Binding domains are further described herein.

An embodiment of the method is illustrated in FIG. 5. In FIG. 5, an oligonucleotide detection reagent comprises, in the order shown, a secondary TAC, a hydrolase cleavage site, a primary TAC, and a detectable label. A hydrolase cleaves one or more copies of the oligonucleotide detection reagent, thereby generating one or more of: (i) first cleaved oligonucleotide detection reagents, each comprising the primary TAC and detectable label; and (ii) cleaved secondary TAC, each comprising the secondary TAC. The reaction mixture sample is incubated on a binding surface comprising a secondary targeting agent to bind the one or more cleaved secondary TAC(s), thereby separating the cleaved secondary TAC(s) from the first cleaved oligonucleotide detection reagent(s). The resulting reaction mixture sample is then incubated on a detection surface comprising a primary targeting agent to immobilize the one or more first cleaved oligonucleotide detection reagents onto the detection surface. In embodiments, the detectable label(s) of the immobilized first cleaved oligonucleotide detection reagent(s) are detected as described herein. In embodiments, the nucleic acid of interest is detected via detection of the immobilized first cleaved oligonucleotide detection reagent(s).

An exemplary protocol for performing the multiplexed method comprises:

1. Preparing the samples comprising the hydrolase enzyme activity or activities of interest. In embodiments, the preparing comprises obtaining the sample and extracting or purifying the hydrolase enzyme. In embodiments, the preparing does not require any extracting or purifying of the sample. In embodiments, the preparing comprises diluting the sample with a suitable buffer. In embodiments, the preparing does not require diluting the sample.

2A. Incubating multiple sample reaction mixtures, comprising the oligonucleotide detection reagents (e.g., about 0.05 nM to about 100 nM, about 0.1 nM to about 80 nM, about 0.2 nM to about 60) nM, about 0.3 nM to about 50 nM, about 0.4 nM to about 40 nM, about 0.1 nM to about 20 nM, or about 0.1 nM to about 10 nM of one or more oligonucleotide detection reagents, each one comprising a unique TAC), and the samples that comprise the multiple hydrolase enzyme activities of interest. In embodiments, each unique primary TAC comprises a unique nucleic acid sequence. In embodiments, the sample reaction mixture is in an assay buffer of pH about 6 to about 8, about 6.5 to about 7.5, or about 6.7 to about 7. In embodiments, the sample reaction mixture comprises a reaction volume of about 10 μL to about 1 mL, about 20 μL to about 700 μL, about 50 μL to about 500 μL, about 70 μL to about 200 μL, about 90 μL to about 150 μL, or about 100 μL. In embodiments, the sample reaction mixture is incubated for about 10 minutes to about 6 hours, about 30 minutes to about 4 hours, or about 1 hour to about 3 hours. In embodiments, the sample reaction mixture is incubated at about 20° C., to about 50° C., about 25° C., to about 45° C., about 30° C., to about 40° C., or about 37° C. In embodiments, the sample reaction mixture is incubated for about 1 hour to about 3 hours at about 37° C.

2B. Preparing an assay plate. In embodiments, the assay plate comprises multiple binding domains in each well, wherein each binding domain comprises a unique primary targeting agent that corresponds to a unique primary TAC on the oligonucleotide detection reagent. In embodiments, each unique primary targeting agent comprises a unique nucleic acid sequence that is complementary to its corresponding primary TAC. In embodiments, the assay plate is a 96-well plate. In embodiments, the assay plate is blocked with a blocking solution. In embodiments, the blocking solution reduces and/or eliminates non-specific binding to the primary targeting agent on the assay plate. In embodiments, following the washing, a hybridization buffer is added to the assay plate (e.g., about 10 μL to about 50 μL, about 20 μL to about 40 μL, or about 30 μL per well of the assay plate). In embodiments, the hybridization buffer facilitates binding of the primary TAC to the primary targeting agent.

In embodiments, steps 1 and 2 are performed simultaneously or substantially simultaneously.

3A. Incubating the sample reaction on the assay plate comprising the hybridization buffer. In embodiments, about 10 μL to about 100 μL, about 20 μL to about 80 μL, about 30 μL to about 70 μL, about 40 μL to about 60 μL, or about 50 μL of the sample reaction is added to a well of the assay plate. In embodiments, the sample reaction is incubated for about 10 minutes to about 4 hours, about 30 minutes to about 2 hours, or about 1 hour. In embodiments, the sample reaction is incubated at about 15° C., to about 40° C., about 20° C., to about 37° C., about 25° C., to about 30° C., or about 27° C. In embodiments, the sample reaction is incubated for about 1 hour at about 27° C. In embodiments, the assay plate is washed, e.g., with PBS, following the incubating.

3B. Removing cleaved secondary TAC and/or uncleaved oligonucleotide detection reagent. In embodiments, the removing comprises contacting the sample reaction with magnetic beads comprising a secondary targeting agent. In embodiments, the secondary targeting agent is a binding partner of a secondary TAC and/or an amplification blocker on the oligonucleotide detection reagent. In embodiments, the magnetic beads are incubated with the sample reactions for about 10 minutes to about 4 hours, about 30 minutes to about 2 hours, or about 1 hour. In embodiments, the magnetic beads are incubated with the sample reaction at about 20° C., to about 50° C., about 25° C., to about 45° C., about 30° C., to about 40° C., or about 37° C. In embodiments, the magnetic beads are incubated with the sample reaction for about 1 hour at about 37° C. In embodiments, following the incubation, the beads are removed or separated (e.g., concentrated on a side of the sample reaction container such that the beads are no longer in contact with the sample reaction) from the sample reaction by contacting the sample reaction container with a magnet.

4. Reading the plate. In embodiments, about 50 μL to about 500 μL, about 100 μL to about 300 μL, or about 150 μL of a read buffer is added to the assay plate well. In embodiments, the assay plate is read on a plate reader immediately or substantially immediately following addition of the read buffer.

### Assays and Diagnostics

In embodiments, the method and kit described herein can be used in assaying for hydrolase enzyme activity associated with a specific outcome. In embodiments, the method and kit described herein can be used to determine the presence of a specific hydrolase enzyme activity in a sample.

In embodiments, the method and kit described herein can be used in a drug development assay. In embodiments, the drug being developed is a hydrolase enzyme inhibitor and the drug development assay is used to determine is the drug inhibits hydrolase enzyme activity. In embodiments, the drug being developed is a protease inhibitor, and the drug development assay is used to determine if the drug inhibits HIV protease enzyme activity. In embodiments, the drug the drug development assay is used to determine if the drug inhibits renin activity for use in blood pressure indications. In embodiments, the drug being developed is a hydrolase enzyme activator and the drug development assay is used to determine is the drug activates hydrolase enzyme activity. In embodiments, the drug development assay is used to determine if the drug inhibits influenza neuraminidase activity.

In embodiments, the method and kit described herein are used in medical diagnosis. In embodiments, a sample is taken from the subject to be diagnosed. Samples are further described herein. In embodiments, the method and kit described herein are used in diagnosing a bone disease, autoimmune disorder, inflammatory disorder, rheumatoid arthritis, arthritis or joint infection. In embodiments, the medical diagnosis is rheumatoid arthritis and the hydrolase enzyme detected is a Cathepsin, a Gelatinase or Lysozyme. In embodiments, the medical diagnosis is rheumatoid arthritis and the hydrolase enzyme detected is a Cathepsin D or Gelatinase B. In embodiments, the medical diagnosis is diabetes or pancreatitis and the hydrolase enzyme detected is an amylase.

In embodiments, the method and kit are used in diagnosing a cancer, e.g., breast cancer, hepatocellular carcinoma, gastric cancer, prostate cancer or germ cell malignancy. In embodiments, the method and kit are used in diagnosing metastasis of a cancer. In embodiments, the method and kit are used in diagnosing a premalignancy. In embodiments for diagnosing a cancer, metastasis or premalignancy, the hydrolase enzyme detected is a Cathepsin. In embodiments for diagnosing a cancer, metastasis or premalignancy, the hydrolase enzyme detected is Cathepsin D. In embodiments for diagnosing acute myelomonocytic leukemia (FAB-M4), chronic myelomonocytic leukemia (CMML) and/or chronic myelocytic leukemia, the hydrolase enzyme detected is lysozyme (in serum or urine).

In embodiments, the method and kit are used in diagnosing diabetes, Gaucher's disease, liver disease, myocardial infarction, pancreatitis, a dental disorder, a kidney disorder, a skin disorder or a neurological disorder. In embodiments, the method and kit are used to detect active PRTN3 levels in sputum, as such levels have been shown to correlate with clinical markers of inflammation in respiratory diseases including cystic fibrosis and chronic obstructive pulmonary disease. In embodiments, the method and kit are used to measure active PRTN3 in sputum to aid in the understanding of several pulmonary diseases. In embodiments, the method and kit are used in diagnosing tuberculosis, sarcoidosis, megaloblastic anemias, acute bacterial infections, ulcerative colitis and Crohn's disease. In embodiments, the method and kit are used in diagnosing severe renal insufficiency, renal transplant rejection, urinary tract infections, glomerulonephritis and nephrosis.

In embodiments, the method and kit described herein can be used in determining the presence of a virus or microorganism is a sample. In embodiments, the method and kit described herein are used to detect hydrolase enzyme activity associated with a virus or microorganism. In embodiments, the sample is taken from a living subject and the detection of hydrolase enzyme activity associated with a virus or microorganism is a diagnosis that the subject is infected with the virus or microorganism.

In embodiments, the method and kit described herein can be used to monitor proteins during purification to ensure proteases are absent.

### Assay Components

### Binding Surface

In embodiments comprising a binding surface, the binding surface comprises a secondary targeting agent immobilized thereon. In embodiments, the secondary targeting agent is indirectly immobilized on the detection surface via a binding pair, e.g., a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the secondary targeting agent and the binding surface comprise cross-reactive moieties, e.g., thiol and maleimide or iodoacetamide: aldehyde and hydrazide: or azide and alkyne or cycloalkyne. In embodiments, the secondary targeting agent comprises biotin, and the binding surface comprises avidin or streptavidin.

In embodiments, the binding surface comprises a planar substrate, e.g., a plate. In embodiments, the binding surface comprises a multi-well plate. In embodiments, the binding surface comprises a particle. In embodiments, the binding surface comprises a magnet. In embodiments where the binding surface comprises a particle, separating the binding surface from a reaction mixture comprises collecting the particle, e.g., via gravity filtration, centrifugation, and/or a magnetic collector, and separating the collected particles from the reaction mixture.

### Detection Surface

In embodiments, the detection surface comprises a targeting agent immobilized thereon. In embodiments, the targeting agent is directly immobilized on the detection surface. In embodiments, the targeting agent is indirectly immobilized on the detection surface via a binding pair, e.g., a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the targeting agent and the detection surface comprise cross-reactive moieties, e.g., thiol and maleimide or iodoacetamide: aldehyde and hydrazide: or azide and alkyne or cycloalkyne. In embodiments, the targeting agent comprises biotin, and the detection surface comprises avidin or streptavidin.

In embodiments, the detection surface comprises a particle. In embodiments, the detection surface comprises a magnetic bead or a magnetic microparticle. In embodiments, the detection surface comprises a microsphere internally dyed with varying proportions of different fluorophores that correspond to a distinct spectral signature, or bead region. In embodiments, the detection surface comprises a particle such as a magnetic bead or microsphere, the detectable label is a fluorescent label, and fluorescence is read on a dual-laser flow-based detection instrument, on a magnetic surface that captures and holds the beads, or on an array imager.

In embodiments, the detection surface comprises a well of multi-well plate. In embodiments, the detection surface comprises a cartridge. In embodiments, the detection surface comprises a plurality of distinct binding domains. In embodiments, each distinct binding domain is positioned about 10 μm to about 100 μm apart from an adjacent distinct binding domain on the detection surface. In embodiments, each distinct binding domain is positioned less than 100 μm apart from an adjacent distinct binding domain on the detection surface. In embodiments, each distinct binding domain is positioned less than 50 μm apart from an adjacent distinct binding domain on the detection surface. In embodiments, each distinct binding domain is positioned less than 10 μm apart from an adjacent distinct binding domain on the detection surface.

In embodiments, the detection surface comprises an electrode. In embodiments, the electrode is a carbon ink electrode. In embodiments, the detecting (e.g., of a detectable label described herein) comprises applying a voltage waveform (e.g., a potential) to the electrode to general an ECL signal. In embodiments, the detection surface comprises a particle, and the method comprises collecting the particle on an electrode and applying a voltage waveform (e.g., a potential) to the electrode to generate an ECL signal.

In embodiments where the method is a multiplexed method for detecting multiple hydrolase enzymes, the detection surface comprises a plurality of binding domains, and each unique nucleic acid of interest is detected in a different binding domain. In embodiments, the detection surface comprises a multi-well plate, and each binding domain is in a different well. In embodiments, the detection surface comprises a well of a multi-well plate, and each binding domain is in a separate portion of the well. In embodiments, the plurality of binding domains is on one or more detection surfaces. In embodiments, the detection surface comprises a particle, and each binding domain is on a different particle. In embodiments, the particles are arranged in a particle array. In embodiments, the particles are coded to allow for identification of specific particles and distinguish between each binding domain.

### Analytes and Samples

In embodiments, the sample is a biological sample. In embodiments, the sample is an environmental sample. In embodiments, the sample is obtained from a human subject. In embodiments, the sample is obtained from an animal subject. In embodiments, the sample comprises a mammalian fluid, secretion, or excretion. In embodiments, the sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, a respiratory sample, bronchial/bronchoalveolar lavage, saliva, mucus, oropharyngeal swab, sputum, endotracheal aspirate, pharyngeal/nasal swab, throat swab, amniotic fluid, nasal secretions, nasopharyngeal wash or aspirate, nasal mid-turbinate swab, vaginal secretions, a surface biopsy, sperm, semen/seminal fluid, wound secretions and excretions, ear secretions or discharge, or an extraction, purification therefrom, or dilution thereof. Further exemplary biological samples include but are not limited to physiological samples, samples containing suspensions of cells such as mucosal swabs, tissue aspirates, endotracheal aspirates, tissue homogenates, cell cultures, and cell culture supernatants. In embodiments, the biological sample is a respiratory sample obtained from the respiratory tract of a subject. Examples of respiratory samples include, but are not limited to, bronchial/bronchoalveolar lavage, saliva, mucus, endotracheal aspirate, sputum, nasopharyngeal/nasal swab, throat swab, oropharyngeal swab and the like. In embodiments, the biological sample is whole blood, serum, plasma, cerebrospinal fluid (CSF), urine, saliva, sputum, endotracheal aspirate, nasopharyngeal/nasal swab, bronchoalveolar lavage, or an extraction or purification therefrom, or dilution thereof. In embodiments, the biological sample is serum or plasma. In embodiments, the plasma is in EDTA, heparin, or citrate. In embodiments, the biological sample is saliva. In embodiments, the biological sample is endotracheal aspirate. In embodiments, the biological sample is a nasal swab.

In embodiments, the sample is an environmental sample. In embodiments, the environmental sample is aqueous, including but not limited to, fresh water, drinking water, marine water, reclaimed water, treated water, desalinated water, sewage, wastewater, surface water, ground water, runoff, aquifers, lakes, rivers, streams, oceans, and other natural or non-natural bodies of water. In embodiments, the aqueous sample contains bodily solids or fluids (e.g., feces or urine) from a human subject.

Samples may be obtained from a single source described herein, or may contain a mixture from two or more sources.

In embodiments, the sample comprises or is suspected to comprise a hydrolase enzyme of interest. Hydrolase enzymes of interest include any biological catalysts, both natural and synthetic, including protein enzymes, nucleic acid enzymes and enzymes comprising both protein and nucleic acid.

In embodiments, the detection limit of the method is about 1 fg/mL to about 10⁶ fg/mL, about 1 fg/mL to about 10⁵ fg/mL, about 1 fg/mL to about 10⁴ fg/mL, about 1 fg/mL to about 1000 fg/mL, or about 1 fg/mL to about 100 fg/mL of the hydrolase enzyme in the sample.

### Assay Devices, Manual and Automated Embodiments

The methods herein can be conducted in a single assay chamber, such as a single well of an assay plate. The methods herein can also be conducted in an assay chamber of an assay cartridge. The assay modules, e.g., assay plates or assay cartridges, methods and apparatuses for conducting assay measurements suitable for the present invention, are described, e.g., in U.S. Pat. Nos. 8,343,526; 9,731,297; 9,921,166; 10,184,884; 10,281,678; 10,272,436; US 2004/0022677; US 2004/0189311; US 2005/0052646; US 2005/0142033: US 2018/0074082; and US 2019/0391170.

The methods herein can be performed manually, using automated technology, or both. Automated technology may be partially automated, e.g., one or more modular instruments, or a fully integrated, automated instrument. Exemplary automated systems and apparatuses are described in WO 2018/017156, WO 2017/015636, and WO 2016/164477.

In embodiments, automated systems, e.g., modular and fully integrated systems, for performing the methods herein comprises one or more of the following automated subsystems: a computer subsystem comprising hardware (e.g., personal computer, laptop, hardware processor, disc, keyboard, display, printer), software (e.g., processes such as drivers, driver controllers, and data analyzers), and/or a database: a liquid handling subsystem for sample and/or reagent handling, e.g., comprising a robotic pipetting hand, syringe, stirring apparatus, ultrasonic mixing apparatus, and/or magnetic mixing apparatus: a sample, reagent, and/or consumable storing and handling subsystem, e.g., comprising a robotic manipulator, tube or lid or foil piercing apparatus, lid removing apparatus, conveying apparatus such as linear or circular conveyor, tube rack, plate carrier, trough carrier, pipet tip carrier, plate shaker, and/or centrifuge: an assay reaction subsystem, e.g., that is fluid-based and/or consumable-based (such as tube and multi-well plate); a container and consumable washing subsystem, e.g., comprising a plate washing apparatus: a magnetic separator or magnetic particle concentrator subsystem, e.g., that is flow cell type, tube type, and/or plate type: a cell and particle detection, classification, and/or separation subsystem, e.g., comprising a flow cytometer and/or a Coulter counter: a detection subsystem, e.g., comprising a colorimetric detector, a nephelometric detector, a fluorescence detector, and/or an ECL detector: a temperature control subsystem, e.g., comprising an air handling system, air cooling system, air warming system, fan, blower, and/or water bath; a waste subsystem, e.g., comprising liquid and/or solid waste containers: a global unique identifier (GUI) detecting subsystem, e.g., comprising 1D and/or 2D barcode scanners such as flat bed and wand type scanners. In embodiments, the automated system further comprises a modular or fully integrated analytical subsystem, e.g., a chromatography system such as high-performance liquid chromatography (HPLC) or fast-protein liquid chromatography (FPLC), or a mass spectrometer.

In embodiments, systems or modules that perform sample identification and

preparation are combined with, adjoined to, adjacent to, and/or robotically linked or coupled to the systems or modules that perform and/or detect the assays herein. Multiple modular systems of the same type can be combined to increase throughput. In embodiments, a modular system is combined with a module that performs other types of analysis, such as chemical, biochemical, and/or nucleic acid analysis.

In embodiments, the automated system allows batch, continuous, random-access, and/or point-of-care workflows, and single, medium, and high sample throughput.

In embodiments, the automated system comprises one or more of the following devices: a plate sealer (e.g., ZYMARK™), a plate washer (e.g., BIOTEK™, TECAN™), a reagent dispenser, automated pipetting station, and/or liquid handling station (e.g., TECAN™, ZYMARK™, LABSYSTEMS™, BECKMAN™, HAMILTON™), an incubator (e.g., ZYMARK™), a plate shaker (e.g., Q.INSTRUMENTS™, INHECO™, THERMOFISHER™), a compound library module, a sample storage module, and/or a compound and/or sample retrieval module. In embodiments, one or more of these devices is coupled to the automated system via a robotic assembly such that the entire assay process can be performed automatically. In embodiments, a container (e.g., a plate) is manually moved between the apparatus and various devices described herein (e.g., a stack of plates).

In embodiments, the automated system is configured to perform one or more of the following functions: moving consumables such as plates into, within, and out of the detection subsystem; moving consumables between other subsystems; storing the consumables; sample and reagent handling (e.g., adapted to mix reagents and/or introduce reagents into consumables); consumable shaking (e.g., for mixing reagents and/or for increasing reaction rates); consumable washing (e.g., washing plates and/or performing assay wash steps (e.g., well aspirating)); measuring a detectable signal, e.g., ECL signal, in a flow cell or a consumable such as a tube or a plate. The automated system may be configured to handle individual tubes placed in racks and/or multi-well plates such as 96 or 384 well plates.

Methods for integrating components and modules in automated systems as described herein are discussed, e.g., by Sargeant et al., “Platform Perfection,” Medical Product Outsourcing, May 17, 2010.

In embodiments, the automated system is fully automated, modular, computerized, performs in vitro quantitative and qualitative tests on a wide range of analytes, and/or performs photometric assays, ion-selective electrode measurements, and/or electrochemiluminescence (ECL) assays. In embodiments, the system comprises one or more of the following hardware units: a control unit, a core unit and at least one analytical module.

In embodiments, the control unit utilizes a graphical user interface to control all instrument functions and comprises a readout device, such as a monitor; an input device, such as key board and mouse; and a personal computer, e.g., using a Windows operating system. In embodiments, the core unit comprises one or more components that manage conveyance of samples to each assigned analytical module. The actual composition of the core unit depends on the configuration of the analytical modules, which can be configured by one of skill in the art using methods known in the art. In embodiments, the core unit comprises at least the sampling unit and one rack rotor as main components. In embodiments, the control unit further comprises an extension unit, e.g., a conveyor line and/or a second rack rotor. In embodiments, the core unit further comprises a sample rack loader/unloader, a port, a barcode reader (for racks and samples), a water supply, and a system interface port. In embodiments, the automated system conducts ECL assays and comprises a reagent area, a measurement area, a consumables area, and a pre-clean area.

### Kits Comprising Oligonucleotide Detection Reagents Comprising a Targeting Agent Blocker

In embodiments, the disclosure provides a kit for detecting a hydrolase enzyme activity of interest, comprising: (a) at least one oligonucleotide detection reagent, wherein the oligonucleotide detection reagent comprises: (i) a targeting agent complement (TAC); (ii) a targeting agent blocker that is complementary to at least a portion of the TAC; and (iii) a hydrolase cleavage site; and (b) one or more detecting reagents for detecting an unblocked oligonucleotide. In embodiments of the kit, the oligonucleotide detection reagent further comprises iv. a detectable label.

In embodiments of the kit, the hydrolase cleavage site comprises a nucleic acid. In embodiments, the hydrolase cleavage site comprises a polypeptide. In embodiments, the hydrolase cleavage site comprises a polysaccharide. In embodiments, the hydrolase cleavage site comprises a lipid. In embodiments of the kit for detecting more than one hydrolase enzyme activity, the kit can comprise more than one oligonucleotide detection reagent, where each oligonucleotide detection reagent comprises a hydrolase cleavage site comprising a nucleic acid, polypeptide, a polysaccharide or a lipid.

Oligonucleotide detection reagents are further described herein. In embodiments, the oligonucleotide detection reagent comprises a single strand. In embodiments, the oligonucleotide detection reagent comprises RNA. In embodiments, the oligonucleotide detection reagent comprises ssDNA. In embodiments, the oligonucleotide detection reagent comprises, in 5′ to 3′ order, the targeting agent blocker, the hydrolase cleavage site, the TAC, and the detectable label. In embodiments, the oligonucleotide detection reagent comprises, in 3′ to 5′ order, the targeting agent blocker, the hydrolase cleavage site, the TAC, and the detectable label.

In embodiments, the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length. In embodiments where the hydrolase cleavage site comprises a peptide, polysaccharide or lipid, the total length of the nucleic acid portions of the oligonucleotide detection reagent is about 20 to about 300, about 25 to about 280, about 30 to about 250), about 35 to about 220, about 40 to about 200, about 45 to about 180, about 50 to about 150, about 55 to about 120, about 60 to about 100, or about 65 to about 80 nucleotides in length. In embodiments, the TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40), or about 30 to about 35 nucleotides in length. In embodiments, the TAC and the targeting agent blocker are substantially the same length. In embodiments, the TAC and the targeting agent blocker differ in length by no more than 1, 2, 3, 4, 5, 6, 7, 8, 9, or 10 nucleotides. In embodiments, the targeting agent blocker is shorter than the TAC by about 1, 2, 3, 4, 5, 6, 7, 8, 9, or 10 nucleotides. In embodiments, the TAC is shorter than the targeting agent blocker by about 1, 2, 3, 4, 5, 6, 7, 8, 9, or 10 nucleotides. In embodiments, the TAC comprises any of SEQ ID NOs: 14-17.

In embodiments where the oligonucleotide detection reagent comprises a single strand, the hydrolase cleavage site is capable of forming an oligonucleotide loop structure, thereby allowing the targeting agent blocker to hybridize to the TAC. In embodiments, the oligonucleotide loop structure is a hairpin loop. In embodiments, the TAC is capable of hybridizing to the targeting agent blocker. In embodiments, the hydrolase cleavage site loop structure is capable of stabilizing the hybridization of the TAC and the targeting agent blocker.

In embodiments, the oligonucleotide detection reagent comprises a double strand. In embodiments, the TAC is on a first strand of the oligonucleotide detection reagent, and the targeting agent blocker and the hydrolase cleavage site are on a second strand of the oligonucleotide detection reagent. In embodiments, the targeting agent blocker comprises a first region and a second region, and the hydrolase cleavage site is positioned between the first region and the second region of the targeting agent blocker; and wherein the first region and the second region of the targeting agent blocker are capable of hybridizing to first and second regions of the TAC, respectively. In embodiments, the first strand of the oligonucleotide detection reagent comprises, in 5′ to 3′ order: the first region of the targeting agent blocker, the hydrolase cleavage site, and the second region of the targeting agent blocker. In embodiments, the second strand of the oligonucleotide detection reagent comprises, in 3′ to 5′ order: the second region of the TAC (which is complementary to the second region of the targeting agent blocker), the first region of the TAC (which is complementary to the first region of the targeting agent blocker), and the detectable label. In embodiments, the first strand of the oligonucleotide detection reagent comprises, in 3′ to 5′ order: the first region of the targeting agent blocker, the hydrolase cleavage site, and the second region of the targeting agent blocker. In embodiments, the second strand of the oligonucleotide detection reagent comprises, in 5′ to 3′ order: the second region of the TAC. the first region of the TAC, and the detectable label. In embodiments, the TAC is capable of hybridizing to the targeting agent blocker. In embodiments, the presence of the hydrolase cleavage site between the first and second regions of the targeting agent blocker is capable of stabilizing the hybridization of the TAC and the targeting agent blocker.

In embodiments, the kit further comprises c) a detection surface comprising a targeting agent, wherein the targeting agent is a binding partner of the TAC, wherein uncleaved oligonucleotide detection reagent does not substantially bind to the detection surface.

In embodiments, the TAC is a binding partner of a targeting agent on the detection surface. In embodiments, the targeting agent and the detection surface are provided separately in the kit, and the kit further comprises a reagent for immobilizing the targeting agent onto the detection surface. In embodiments, the TAC and the targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and TAC are cross-reactive moieties, e.g., thiol and maleimide or iodoacetamide: aldehyde and hydrazide: or azide and alkyne or cycloalkyne. In embodiments, the TAC and the targeting agent comprise complementary oligonucleotides. Targeting agents and TACs are further described herein.

In embodiments, portions of the oligonucleotide detection reagent that are not part of the hydrolase cleavage site comprise nuclease resistant nucleotides. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments of the kit, the detecting reagents comprise one or more hybridization buffers as further described herein. In embodiments, the detecting reagents comprise PCR extension buffers and free nucleotides as further described herein.

In embodiments, the kit comprises at least two, three, four, five, six, seven, eight, nine, ten or more oligonucleotide detection reagents. In embodiments, each hydrolase cleavage site of the two or more oligonucleotide detection reagents comprises a nucleic acid, a peptide, a polysaccharide or combinations thereof.

In embodiments where the oligonucleotide detection reagent comprises a detectable label, the detectable label is detectable by light scattering, optical absorbance, fluorescence, chemiluminescence, electrochemiluminescence (ECL), bioluminescence, phosphorescence, radioactivity, magnetic field, or combinations thereof. In embodiments, the detectable label comprises an ECL label. ECL labels are further described herein.

In embodiments, a portion of the TAC may be used as a primer in an extension reaction. In embodiments, the TAC comprises a primer for polymerase chain reaction (PCR), ligase chain reaction (LCR), strand displacement amplification (SDA), self-sustained synthetic reaction (3SR), or an isothermal amplification method. In embodiments, the primer comprises a primer for an isothermal amplification method. In embodiments, the isothermal amplification method is helicase-dependent amplification. In embodiments, the isothermal amplification method is rolling circle amplification (RCA). In embodiments, the primer is about 10 to about 50, about 11 to about 45, about 12 to about 40, about 13 to about 35, about 14 to about 30, about 15 to about 40, about 20 to about 35, about 25 to about 30, or about 30 to about 35 nucleotides in length. In embodiments, the primer is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

### Kits Comprising Oligonucleotide Detection Reagents Comprising a Secondary Targeting Agent

In embodiments, the invention provides a kit for detecting a hydrolase enzyme, comprising, in one or more containers, vials, or compartments: (a) an oligonucleotide detection reagent that comprises (i) a primary targeting agent complement (primary TAC): (ii) a secondary targeting agent complement (secondary TAC): (iii) a hydrolase cleavage site and (iv) a detectable label; and (b) one or more detecting reagents for detecting a primary TAC. In embodiments, the kit further comprises one or both of a binding surface comprising a secondary targeting agent and a detection surface comprising a primary targeting agent.

In embodiments of the kit, the hydrolase cleavage site comprises a nucleic acid. In embodiments, the hydrolase cleavage site comprises a polypeptide. In embodiments, the hydrolase cleavage site comprises a polysaccharide. In embodiments, the hydrolase cleavage site comprises a lipid. In embodiments of the kit for detecting more than one hydrolase enzyme activity, the kit can comprise more than one oligonucleotide detection reagent, where each oligonucleotide detection reagent comprises a hydrolase cleavage site comprising a nucleic acid, polypeptide, a polysaccharide or a lipid.

Oligonucleotide detection reagents are further described herein. In embodiments, the oligonucleotide detection reagent comprises, in the order listed: a secondary TAC, a hydrolase cleavage site, a primary TAC, and a detectable label. In embodiments, a hydrolase enzyme cleaves the hydrolase cleavage site, thereby generating one or more of: (i) first cleaved oligonucleotide detection reagent, each comprising the primary TAC and detectable label; and (ii) cleaved secondary TACs, each comprising the secondary TAC. In embodiments, the secondary TAC, hydrolase cleavage site, primary TAC and detectable label are arranged as shown in FIG. 5.

In embodiments, the oligonucleotide detection reagent is DNA. In embodiments, the oligonucleotide detection reagent is RNA. In embodiments, the oligonucleotide detection reagent is a combination of DNA and RNA. In embodiments, the oligonucleotide detection reagent is a nucleic acid with a non-natural backbone. As discussed in further detail herein, in embodiments, the hydrolase cleavage site in the oligonucleotide detection reagent can comprise a peptide, polysaccharide or lipid. In embodiments where the hydrolase cleavage site comprises a peptide, polysaccharide or lipid, the nucleic acid portions of the oligonucleotide detection reagent are DNA. In embodiments where the hydrolase cleavage site comprises a peptide, polysaccharide or lipid, the nucleic acid portions of the oligonucleotide detection reagent are RNA. In embodiments where the hydrolase cleavage site comprises a peptide, polysaccharide or lipid, the nucleic acid portions of the oligonucleotide detection reagent comprise a nucleic acid with a non-natural backbone. In embodiments, portions of the oligonucleotide detection reagent that are not part of the hydrolase cleavage site comprise nuclease resistant nucleotides. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety. or a combination thereof.

In embodiments, the secondary TAC is a binding partner of a secondary targeting agent on a binding surface. In embodiments, the secondary targeting agent and the binding surface are provided separately in the kit, and the kit further comprises a reagent for immobilizing the secondary targeting agent onto the binding surface. In embodiments, the secondary TAC and the secondary targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the secondary targeting agent and secondary TAC are cross-reactive moieties, e.g., thiol and maleimide or iodoacetamide: aldehyde and hydrazide: or azide and alkyne or cycloalkyne. In embodiments, the secondary TAC comprises biotin, and the secondary targeting agent comprises avidin or streptavidin. In embodiments, the secondary TAC is an oligonucleotide of about 5 to about 100, about 6 to about 90, about 7 to about 80, about 8 to about 70, about 9 to about 60) nucleotides, about 10 to about 50), about 15 to about 45, about 20 to about 40, about 20 to about 30, or about 30 to about 35 nucleotides in length. Secondary TACs, secondary targeting agents, and binding surfaces are further described herein.

In embodiments, the primary TAC is a binding partner of a primary targeting agent on a detection surface. In embodiments, the targeting agent and the detection surface are provided separately in the kit, and the kit further comprises a reagent for immobilizing the targeting agent onto the detection surface. In embodiments, the primary TAC and the primary targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the primary targeting agent and the primary TAC are cross-reactive moieties, e.g., thiol and maleimide or iodoacetamide: aldehyde and hydrazide: or azide and alkyne or cycloalkyne. In embodiments, the primary TAC and the primary targeting agent comprise complementary oligonucleotides. In embodiments, the primary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, about 20 to about 35, or about 30 to about 35 nucleotides in length. In embodiments, the primary TAC comprises any of SEQ ID NOs: 14-17. In embodiments, the primary TAC and the primary targeting agent are substantially unreactive with the secondary TAC and the secondary targeting agent. Primary targeting agents, TACs, and detection surfaces are further described herein.

In embodiments of the kit, the detecting reagents comprise one or more hybridization buffers as further described herein.

In embodiments, the kit comprises at least two, three, four, five, six, seven, eight, nine, ten or more oligonucleotide detection reagents. In embodiments, each hydrolase cleavage site of the two or more oligonucleotide detection reagents comprises a nucleic acid, a peptide, a polysaccharide or combinations thereof.

In embodiments, the detectable label is detectable by light scattering, optical absorbance, fluorescence, chemiluminescence, electrochemiluminescence (ECL), bioluminescence, phosphorescence, radioactivity, magnetic field, or combinations thereof. In embodiments, the detectable label comprises phycoerythrin (PE). In embodiments, the detectable label comprises a β-galactosidase (β-gal) enzyme that can be detected by fluorescence detection when the β-gal enzyme cleaves a substrate such as resorufin-β-D-galactopyranoside to yield a fluorescent signal. In embodiments, the detectable label comprises an ECL label. ECL labels are further described herein.

### Kit Components

In embodiments, the detection surface of the kit comprises a particle. In embodiments, the detection surface comprises a well of multi-well plate. In embodiments, the detection surface comprises a cartridge. In embodiments, the detection surface comprises an electrode, e.g., for generating an electrochemiluminescence signal as described herein. In embodiments, the electrode is a carbon ink electrode. In embodiments, the kit further comprises a particle array.

In embodiments where the kit comprises a binding surface, the binding surface comprises a planar substrate. In embodiments, the binding surface comprises a multi-well plate. In embodiments, the binding surface comprises a particle. In embodiments, the binding surface comprises a magnet. In embodiments, the kit further comprises a device for separating and/or collecting the binding surface from a reaction mixture.

In embodiments, the components of the kit, e.g., oligonucleotide binding reagent, oligonucleotide detection reagent, targeting agent, secondary targeting agent, hydrolase, or a combination thereof, are provided lyophilized. In embodiments, the components of the kit, e.g., oligonucleotide binding reagent, oligonucleotide detection reagent, targeting agent, secondary targeting agent, hydrolase, or a combination thereof, are provided in solution. In embodiments, each component of the kit is provided in a separate container, vial, or compartment. In embodiments, each component of the kit is provided separately according to its optimal shipping or storage temperature.

In embodiments, the kit further comprises a calibration reagent. In embodiments, the calibration reagent comprises a known quantity of a control nucleic acid. In embodiments, the kit further comprises a control oligonucleotide binding reagent that comprises a hybridization region complementary to the control nucleic acid. In embodiments, the kit further comprises a control oligonucleotide detection reagent is that known to be collaterally cleaved by the hydrolase upon binding and/or cleavage of the control nucleic acid. In embodiments, the kit comprises multiple calibration reagents comprising a range of concentrations of the control nucleic acid. In embodiments, the multiple calibration reagents comprise concentrations of the control nucleic acid near the upper and lower limits of quantitation for the method. In embodiments, the multiple calibration reagents span the entire dynamic range of the method. In embodiments, the calibration reagent is a positive control reagent. In embodiments, the calibration reagent is a negative control reagent. In embodiments, the positive or negative control reagent is used to provide a basis of comparison for the sample to be assayed with the methods of the present invention.

In embodiments, the kit further comprises a polymerase, a ligase, a labeled probe, a buffer, a co-reactant, a blocking agent, a diluent, a stabilizing agent, a calibration agent, an assay consumable, an electrode, or a combination thereof.

In embodiments, the kit further comprises a polymerase, e.g., for performing polymerase chain reaction (PCR), ligase chain reaction (LCR), strand displacement amplification (SDA), self-sustained synthetic reaction (3SR), and/or isothermal amplification (such as, e.g., helicase-dependent amplification or rolling circle amplification). In embodiments, the kit further comprises a ligase, e.g., for ligating the template oligonucleotide.

In embodiments, the kit further comprises a labeled probe. In embodiments, the labeled probe comprises a detectable label and a complementary sequence to an extended sequence as described herein. Detectable labels are further described herein. In embodiments, the detectable label is an ECL label.

In embodiments, the kit further comprises a buffer, e.g., an assay buffer, a hybridization buffer, a reconstitution buffer, a wash buffer, a storage buffer, a read buffer, or a combination thereof. Hybridization buffer that can be used to provide the appropriate conditions (e.g., stringent conditions) for hybridization of complementary oligonucleotides. In embodiments, the hybridization buffer includes a nucleic acid denaturant such as formamide. In embodiments, the hybridization buffer is provided as two separate components that can be combined to form the hybridization buffer.

In embodiments, the kit further comprises a read buffer comprising a co-reactant, e.g., for performing an electrochemiluminescence measurement. Exemplary co-reactants are described, e.g., in WO 2020/142313 and U.S. Pat. Nos. 6,919,173; 7,288,410; 7,491,540; and 8,785,201.

In embodiments, the kit further comprises a blocking agent, e.g., to decrease non-specific interactions or assay signals from components in the sample that may interfere with the methods described herein. In embodiments, the kit further comprises a diluent for one or more components of the kit. In embodiments, a kit comprising the components above includes stock concentrations of the components that are 5×, 10×, 20×, 30×, 40×, 50×, 60×, 70×, 80×, 90×, 100×, 125×, 150× or higher fold concentrations of a working concentration for the methods provided herein. In embodiments, the kit further comprises a stabilizing agent, e.g., for storage of one or more components of the kit.

In embodiments, the kit further comprises an assay consumable, e.g., assay modules, vials, tubes, liquid handling and transfer devices such as pipette tips, covers and seals, racks, labels, and the like. In embodiments, the kit further comprises an assay instrument and/or instructions for carrying out the methods described herein.

All references cited herein, including patents, patent applications, papers, textbooks and the like, and the references cited therein, to the extent that they are not already, are hereby incorporated herein by reference in their entirety.

### EXAMPLES

### Example 1—Assay for RNase I Activities Using Oligonucleotide Detection Reagents Having Nucleic Acid Hydrolase Cleavage Sites

Oligonucleotide detection reagents where the targeting agent complement, the hydrolase cleavage site, the targeting agent blocker and a detectable label are comprised on a single strand are described above and depicted in FIG. 2. Oligonucleotide detection reagents having a hydrolase cleavage site comprising RNA that is cleaved by RNase I are used in the assays. Exemplary oligonucleotide detection reagent sequences for detection of RNase I activity are shown in Table 1.

TABLE 1
Oligonucleotide Detection Reagent Sequences for Detecting RNase I Activity

| ID Name | Sequence | Notes |
|---|---|---|
| ECL RNase I | ATCATGTCTGGGTTAC rUrUrUrUrUrUrUrUrUrU | Poly rU |
| HP1 | ACTGGTAACCCAGACATGATCGGT/3AmMO/ | cleavage site |
| | (SEQ ID NO: 1) | Targeting |
| ECL RNase I | CATGTCTGGGTTAC rUrUrUrUrU | agent |
| HP2 | ACTGGTAACCCAGACATGATCGGT/3AmMO/ | complement |
| | (SEQ ID NO: 2) | (TAC) |
| ECL RNase I | TCATGTCTGGGTTA rUrUrUrUrU | sequence 1 |
| HP3 | ACTGGTAACCCAGACATGATCGGT/3AmMO/ | |
| | (SEQ ID NO: 3) | |
| ECL RNase I | TCATGTCTGGGTTA rUrUrUrUrUrUrUrUrUrU | |
| HP4 | ACTGGTAACCCAGACATGATCGGT/3AmMO/ | |
| | (SEQ ID NO: 4) | |

For the oligonucleotide detection reagents shown in Table 1, the portion in italics is the targeting agent blocker, the hydrolase cleavage site is the poly rU sequence and the underlined portion is the targeting agent complement (TAC). The oligonucleotide detection reagents have a 3′ amino modification (/3AmMO/) that is conjugated to STAG-NHS (Meso Scale Diagnostics, Rockville, MD) for labeling following standard methods, followed by column chromatography purification, to yield the labeled oligonucleotide detection reagents shown.

To perform a multiplexed assay with four samples, four individual reactions are prepared, each with an ECL-labeled oligonucleotide detection reagent specific for a different binding domain on an assay surface, wherein each binding domain includes a targeting agent sequence that is complementary to a unique TAC sequence (e.g., TAC sequence 1, 3, 8, or 10 as shown in Table 1). This allows each of the reactions to be pooled for ECL analysis, providing a greater degree of multiplexing.

Each 100 μL reaction contains from 10 nM to 45 nM purified RNAase I or a sample containing RNase I, 22.5 nM crRNA, 4 nM to 40 nM oligonucleotide detection reagents shown in Table 1 (0.2 to 2 pmoles/well) in reaction buffer (100 mM NaCl, 50 mM Tris-HCl, 10 mM MgCl₂, 1 mM DTT (pH 7.9 (@ 25° C.)). Reactions are allowed to proceed for 1 to 3 hours at 37° C. Purified RNases I is available commercially (New England Biolabs, MA).

During the RNase incubation, an assay plate with multiple binding domains as described above is blocked for 1 hour with a blocking solution, followed by washing and addition of 30 μL of a hybridization buffer.

Following the incubation of the samples with RNase I, 50 μg of magnetic beads (e.g., DYNABEADS™ M-270 Streptavidin (ThermoFisher)) in 15 μL of 100 mM EDTA, 1% SDS are added to bind the uncleaved oligonucleotide detection reagents. This mixture is incubated for 1 hour at room temperature with shaking.

Following the incubation of the samples with the magnetic beads, the beads are removed from the sample reaction mixtures removed or concentrated on the side of the reaction tubes by contacting the tubes with a magnet. Following the magnetic separation, 20 μL of each of the four reactions are added to the hybridization buffer in the blocked assay plates described above and incubated for 1 hour at 37° C.

Following the assay plate incubation, the plate is washed, 150 μL of read buffer is added (e.g., MSD GOLDR Read Buffer B (Meso Scale Discovery)), and the plate is read on a plate reader (e.g., MSD SECTOR® 6000 Reader (Meso Scale Discovery)). Detection of signal indicates that the TAC has hybridized with the targeting agent, indicating hydrolase activity.

### Example 2—Assay for RNase T1 and RNase a Activities Using Oligonucleotide Detection Reagents Having Nucleic Acid Hydrolase Cleavage Sites

Oligonucleotide detection reagents having a hydrolase cleavage site comprising RNA that is cleaved by RNase T1 or RNase A are used in the assays. The oligonucleotide detection reagents have similar structure to those used in Example 1. An exemplary oligonucleotide detection reagent sequence for detection of RNase T1 or RNase A activity is shown in Table 2.

TABLE 2
Oligonucleotide Detection Reagent Sequences
for Detecting RNase T1 or RNase A Activity

| ID Name | Sequence | Notes |
|---|---|---|
| ECL RNase | TCATGTCTGGGTTA rUrGrUrGrUrGrU | Poly rUrG |
| T1 HP | ACTGGTAACCCAGACATGATCGGT/3AmMO/ | cleavage site |
| | (SEQ ID NO: 5) | Targeting |
| | | agent |
| | | complement |
| | | (TAC) |
| | | sequence 1 |

For the oligonucleotide detection reagent shown in the Table 2, the portion in italics is the targeting agent blocker, the hydrolase cleavage site is the poly rUrG sequence and the underlined portion is the targeting agent complement (TAC). The oligonucleotide detection reagents have a 3′ amino modification (/3AmMO/) that is conjugated to STAG-NHS (Meso Scale Diagnostics, Rockville, MD) for labeling following standard methods, followed by column chromatography purification, to yield the labeled oligonucleotide detection reagents shown.

To perform a multiplexed assay with four samples, four individual reactions are prepared, each with an ECL-labeled oligonucleotide detection reagent specific for a different binding domain on an assay surface, wherein each binding domain includes a targeting agent sequence that is complementary to a unique TAC sequence (e.g., TAC sequence 1, 3, 8, or 10 as shown in Table 1). This allows each of the reactions to be pooled for ECL analysis, providing a greater degree of multiplexing.

Each 100 μL reaction contains from 10 nM to 45 nM purified RNase T1 and RNase A or a sample containing RNase T1 and RNase A, 22.5 nM crRNA, 4 nM to 40 nM oligonucleotide detection reagents shown in Table 1 (0.2 to 2 pmoles/well) in reaction buffer (100 mM NaCl, 50 mM Tris-HCl, 10 mM MgCl₂, 1 mM DTT (pH 7.9 (@) 25° C.)). Reactions are allowed to proceed for 1 to 3 hours at 37° C. Both purified RNase T1 and RNase A are available commercially (e.g., from New England Biolabs, MA).

Assays are performed and detected using the protocol described in Example 1.

### Example 3—Detection of Proteases Using Multiplex Based Substrates

Proteases can be used to generate electrochemiluminescent (ECL) labeled substrates for detection, including the potential to use arrays of immobilized oligonucleotides. This approach offers the potential to multiplex the detection of differing proteases based on their substrate specificities and/or to profile differing substrates for a given protease. Substrate profiling may also be used to screen mutant proteases to evaluate changing specificity either in the context of a protein engineering effort or in the contexts of viral surveillance of emerging viral protease variants. Additionally, the use of multiplex oligonucleotide arrays allows for sample pooling following the protease reaction with its target, this sample pooling allows for multiplexing either samples or analytes. The oligonucleotide detection reagents that act as protease substrates include a specific peptide element, the substrate for the protease, flanked by biotin (a secondary targeting agent complement) and a nucleic acid sequence (a primary targeting agent complement) labeled with an ECL label. The biotin group allows for the removal of uncut substrate to allow the specific capture of ECL label following cutting, and detection of the protease activity via ECL. The oligonucleotide detection reagent protease substrates used in this example have the following basic structure: Biotin-Peptide-Nucleic acid-ECL label.

The Peptide and nucleic acid elements may be arranged in a number or orientations including:
5. Biotin N-Peptide-C-5′-oligonucleotide-3′-ECL
6. Biotin C-Peptide-N-5′-oligonucleotide-3′-ECL
7. Biotin N-Peptide-C-3′-oligonucleotide-5′-ECL
8. Biotin C-Peptide-N-3′-oligonucleotide-5′-ECL

Substrates were made by the conjugation of synthetic peptides (Thermo Fisher, Inc.) to synthetic oligonucleotide (Integrated DNA Technologies) using succinimidyl 4-[N-maleimidomethyl]cyclohexane-1-carboxylate (SMCC) or similar cross linking agents (Thermo Fisher). For conjugation using a cross-linker, the peptide has a N-terminal biotin and a C-terminal Lys and oligonucleotide has a 5′ Thiol and 3′ Amino group. The peptide is reacted with SMCC, to modify the Lys residue and introduce the thiol reactive maleimide group. The oligonucleotide is deprotected using DTT to release the 5′ Thiol, followed by a desalting spin column to remove the DTT and deprotecting group. The purified and activated peptide is further purified via HPLC and added to the deprotected oligonucleotide and reacted with the activated peptide resulting in conjugation of the peptide to oligonucleotide. STAG-NHS is added to label the 3′ amino group of the oligonucleotide, to form the following protease substrate Biotin N-Peptide-C-5′-oligonucleotide-3′-ECL. This labeled substrate is purified via HPLC to remove contaminating reaction products and used for the detection and analysis of proteases following the protocol below.

Examples of protease substrates specific for differing proteases and primary TAC sequences are shown in Table 3.

TABLE 3
Oligonucleotide Detection Reagent Peptide/Nucleic Acid Sequences for Detecting
Protease Activity

| | | | PEPTIDE | |
|---|---|---|---|---|
| | ID | | SEQ ID | |
| Protease | Name | Sequence | NO | Notes |
| Serine | ECL | Biotin-Tyr-Tyr-Abu-Asn-Glu-Pro-Tyr-Lys-(SMCC | 6 | Primary |
| protease | PR3 | linkage)-/5ThioMC6-D/ | | Targeting |
| PR3 | | ACTGGTAACCCAGACATGATCGGT/3AmMO/STAG | | agent |
| (PRTN3) | | | | complement |
| from | | | | |
| neutrophils | | | | |
| Caspase 3, | ECL | Biotin- GDEVDGV-Lys-(SMCC linkage)-/5ThioMC6-D/ | 7 | (TAC) |
| 7, 2 | Casp3 | ACTGGTAACCCAGACATGATCGGT/3AmMO/STAG | | sequence 1 |
| Caspases 1, | ECL | Biotin-GWEHDGV-Lys-(SMCC linkage)-/5ThioMC6-D/ | 8 | Primary |
| 4, 5 | Casp1 | CTAATAGCTCCTGTGCCCTCGTAT /3AmMO/ | | Targeting |
| | RCA | Biotin-GWEHDGVGWEHDGV-Lys-(SMCC linkage)-/ | | agent |
| | Casp1- | 5ThioMC6-D/CTAATAGCTCCTGTGCCCTCGTAT/ | 9 | complement |
| | 1 | 3AmMO/ | | (TAC) |
| | | | | sequence 3 |
| Caspase 6, | ECL | Biotin- GVEHDGV-Lys-(SMCC linkage)-/5ThioMC6-D/ | 10 | Primary |
| 8 and 9 | Casp6 | AATCCGTCGACTAGCCTGAGAATT /3AmMO/ | | Targeting |
| | RCA | Biotin- GVEHDGV GVEHDGV-Lys-(SMCC linkage)-/ | 11 | agent |
| | Casp6- | 5ThioMC6-D/AATCCGTCGACTAGCCTGAGAATT/ | | complement |
| | 1 | 3AmMO/ | | (TAC) |
| | | | | sequence 8 |
| Sequence | ECL | Biotin-Ile-Glu-Gly-Arg-Lys-(SMCC linkage)-/ | 12 | Primary |
| specific | FXa | 5ThioMC6-D/CGTACCATTGAATCTGGAGACCTT/ | | Targeting |
| clotting | | 3 AmMO/ | | agent |
| factor | RCA | Biotin-Ile-Asp-Gly-Arg-Lys-(SMCC linkage)-/ | 13 | complement |
| Factor Xa | FXa-1 | CGTACCATTGAATCTGGAGACCTT/3AmMO/ | | (TAC) |
| protease | | | | sequence 10 |

The primary TAC sequences used in the constructs shown in Table 3 are:

TAC 1-
(SEQ ID NO: 14)
ACTGGTAACCCAGACATGATCGGT
TAC 3-
(SEQ ID NO: 15)
CTAATAGCTCCTGTGCCCTCGTAT
TAC 8-
(SEQ ID NO: 16)
AATCCGTCGACTAGCCTGAGAATT
TAC 10-
(SEQ ID NO: 17)
CGTACCATTGAATCTGGAGACCTT

For the oligonucleotide detection reagents shown in the Table 3, the protease cleavage site is the peptide sequence and the underlined portion is the primary targeting agent complement (TAC). The oligonucleotide detection reagents have a 3′ amino modification (/3 AmMO/) that is conjugated to STAG-NHS (Meso Scale Diagnostics, Rockville, MD) for labeling following standard methods, as discussed above.

### Protease Assays

To perform a multiplexed assay with multiple samples, individual reactions are prepared, each with an ECL-labeled oligonucleotide detection reagent specific for a different binding domain on an assay surface, wherein each binding domain includes a primary targeting agent sequence that is complementary to a unique primary TAC sequence (e.g., primary TAC sequence 1, 3, 8, or 10 as shown in Table 1). This allows each of the reactions to be pooled for ECL analysis, providing a greater degree of multiplexing.

Samples are added to 100 μl containing, 4 to 40 nM of the oligonucleotide detection reagents shown in Table 3 (2 to 0.2 pmoles/well), assay buffer (20 mM HEPES, or 20 mM Tris-HCl, pH 7.5 or other suitable protease buffers see above). Reactions are allowed to proceed for 1-3 hr at 37° C.

During this protease incubation step an assay plate with multiple binding domains as described above is blocked for 1 hour with using MSD N-PLEX blocking solution, followed by washing and addition of 30 μl of hybridization buffer.

Following the incubation of sample, 50 μg of magnetic beads (e.g., DYNABEADS™ M-270 Streptavidin (ThermoFisher)) in 15 μl of 100 mM EDTA, 1% SDS and protease inhibitor cocktail (Millipore Sigma), are added to bind the uncleaved oligonucleotide detection reagents via the secondary TAC (biotin) and inactivate the proteases. This mixture is incubated for 1 hr at room temperature with shaking.

Following the incubation of samples with the magnetic beads, the beads are removed from the sample reaction mixtures removed or concentrated on the side of the reaction tubes by contacting the tubes with a magnet. Following magnetic separation, 20 μl of sample are added to hybridization buffer in the blocked assay plates described above and incubated for 1 hr at 37° C.

Following the assay plate incubation, the plate is washed, 150 μL of read buffer is added (e.g., MSD GOLD® Read Buffer B (Meso Scale Discovery)), and the plate is read on a plate reader (e.g., MSD SECTOR® 6000 Reader (Meso Scale Discovery)). Detection of signal indicates that the TAC has hybridized with the targeting agent, indicating hydrolase activity.

What is claimed is:

1. A method for detecting a hydrolase enzyme in a sample, comprising:
a. contacting the sample with an oligonucleotide detection reagent, wherein the oligonucleotide detection reagent comprises:
i. a targeting agent complement;
ii. a targeting agent blocker that is complementary to at least a portion of the targeting agent complement; and
iii. a hydrolase cleavage site;
wherein the targeting agent complement and the targeting agent blocker are hybridized,
wherein the hydrolase cleaves the oligonucleotide detection reagent at the hydrolase cleavage site, thereby (i) destabilizing hybridization of the targeting agent complement and the targeting agent blocker and (ii) generating an unblocked oligonucleotide comprising the targeting agent complement; and
(b) detecting the unblocked oligonucleotide, thereby detecting the hydrolase enzyme in the sample.

2. The method of claim 1, wherein the targeting agent complement and the targeting agent blocker are on a contiguous strand of the oligonucleotide detection reagent.

3. The method of claim 1 or 2, wherein the oligonucleotide detection reagent further comprises a detectable label.

4. The method of claim 3, wherein the oligonucleotide detection reagent comprises, in 5′ to 3′ order, the targeting agent blocker, the hydrolase cleavage site, the targeting agent complement, and the detectable label.

5. The method of claim 1, wherein the targeting agent complement is on a first strand of the oligonucleotide detection reagent, and the targeting agent blocker and the hydrolase cleavage site are on a second strand of the oligonucleotide detection reagent.

6. The method of claim 5, wherein the targeting agent blocker comprises a first region and a second region, wherein the hydrolase cleavage site is positioned between the first region and the second region; and wherein the first region and the second region of the targeting agent blocker hybridize to first and second regions of the targeting agent complement on the second strand.

7. The method of any one of claims 1 to 4, wherein the targeting agent complement, the hydrolase cleavage site and the targeting agent blocker are comprised on a single strand.

8. The method of any one of claims 1 to 7, wherein the hydrolase enzyme is a nuclease and the hydrolase cleavage site comprises a nucleic acid that is cleaved by the nuclease.

9. The method of any one of claims 1 to 7, wherein the hydrolase enzyme is a peptidase and the hydrolase cleavage site comprises a peptide that is cleaved by the peptidase.

10. The method of any one of claims 1 to 7, wherein the hydrolase enzyme is a glycoside hydrolase and the hydrolase cleavage site comprises a polysaccharide that is cleaved by the glycoside hydrolase.

11. The method of any one of claims 1 to 10, wherein the method further comprises, prior to step (b), immobilizing the unblocked oligonucleotide to a detection surface comprising a targeting agent, wherein the targeting agent is a binding partner of the targeting agent complement, wherein uncleaved oligonucleotide detection reagent does not substantially bind to the detection surface.

12. The method of claim 11, wherein detecting the unblocked oligonucleotide comprises detecting if the unblocked oligonucleotide is bound to the detection surface.

13. The method of any one of claims 1 to 10, wherein detecting the unblocked oligonucleotide comprises:
i. using the unblocked oligonucleotide as a primer in an extension reaction to form an extension product; and
ii. detecting the extension product.

14. The method of claim 13, wherein the extension product is detected by hybridizing the extension product to a complementary sequence comprising a detectable label.

15. The method of claim 13, wherein the extension product is detected by hybridization to an array.

16. The method of any one of claim 11 or 12, wherein hybridization of the targeting agent complement with the targeting agent blocker substantially prevents binding of the targeting agent complement to the targeting agent on the detection surface.

17. The method of any one of claim 11, 12 or 16, wherein the targeting agent complement and the targeting agent comprise complementary oligonucleotides, and the immobilizing comprises hybridizing the targeting agent complement of the unblocked oligonucleotide to the targeting agent on the detection surface.

18. The method of any one of claims 3 to 17, wherein the detectable label is detected by using light scattering, optical absorbance, fluorescence, chemiluminescence, electrochemiluminescence (ECL), bioluminescence, phosphorescence, radioactivity, magnetic field, or combinations thereof.

19. The method of claim 18, wherein the detectable label is an electrochemiluminescent label, and the detecting comprises measuring an ECL signal.

20. The method of any one of claims 1 to 19, wherein the sample is contacted with one or more additional copies of the oligonucleotide detection reagent.

21. The method of claim 20, comprising immobilizing the plurality of unblocked oligonucleotides to the detection surface and detecting the plurality of unblocked oligonucleotides.

22. The method of any one of claims 11, 12 or 16 to 21, wherein the detection surface comprises a particle.

23. The method of any one of claims 11, 12 or 16 to 21, wherein the detection surface comprises a well of a multi-well plate.

24. The method of any one of claims 11, 12 or 16 to 23, wherein the detection surface comprises an electrode.

25. The method of any one of claims 1 to 24 wherein the method is a multiplexed method for detecting one or more hydrolase enzymes in a sample,
wherein step (a) comprises contacting the sample with a plurality of oligonucleotide detection reagents, wherein each oligonucleotide detection reagent comprises a targeting agent complement, a targeting agent blocker hybridized to the targeting agent complement, a hydrolase cleavage site, and a detectable label,
wherein, for each hydrolase enzyme in the sample, a hydrolase cleaves an oligonucleotide detection reagent comprising a unique hydrolase cleavage site for the hydrolase,
thereby generating a plurality of unblocked oligonucleotides, wherein each unblocked oligonucleotide comprises a unique targeting agent complement;
wherein, prior to step (b), the plurality of unblocked oligonucleotides are immobilized to a detection surface comprising a plurality of targeting agents, wherein each targeting agent is a binding partner of a unique targeting agent complement, and wherein uncleaved oligonucleotide detection reagent does not substantially bind to the detection surface; and
wherein step (b) comprises detecting the plurality of unblocked oligonucleotides bound to the detection surface,
thereby detecting the one or more hydrolase enzymes in in the sample.

26. The method of claim 25, wherein each unique targeting agent complement comprises a unique oligonucleotide sequence.

27. The method of claim 25 or 26, wherein each hydrolase enzyme is a nuclease and the hydrolase cleavage site of each oligonucleotide detection reagent comprises a nucleic acid.

28. The method of claim 25 or 26, wherein each hydrolase enzyme is a peptidase and the hydrolase cleavage site of each oligonucleotide detection reagent comprises a peptide.

29. The method of claim 25 or 26, wherein each hydrolase enzyme is a glycoside hydrolase and the hydrolase cleavage site of each oligonucleotide detection reagent comprises a polysaccharide.

30. The method of claim 25 or 26, wherein the sample comprises at least two hydrolases, and wherein the hydrolases are a nuclease, a peptidase, a glycoside hydrolase or combinations thereof.

31. An oligonucleotide detection reagent comprising:
i. a targeting agent complement;
ii. a targeting agent blocker that is complementary to at least a portion of the targeting agent complement; and
iii. a hydrolase cleavage site;
wherein the targeting agent complement and the targeting agent blocker are hybridized.

32. The oligonucleotide detection reagent of claim 31, wherein the targeting agent complement and the targeting agent blocker are on a contiguous strand of the oligonucleotide detection reagent.

33. The oligonucleotide detection reagent of claim 31 or 32, wherein the oligonucleotide detection reagent further comprises a detectable label.

34. The oligonucleotide detection reagent of claim 33, wherein the oligonucleotide detection reagent comprises, in 5′ to 3′ order, the targeting agent blocker, the hydrolase cleavage site, the targeting agent complement, and the detectable label.

35. The oligonucleotide detection reagent of claim 31, wherein the targeting agent complement is on a first strand of the oligonucleotide detection reagent, and the targeting agent blocker and the hydrolase cleavage site are on a second strand of the oligonucleotide detection reagent.

36. The oligonucleotide detection reagent of claim 35, wherein the targeting agent blocker comprises a first region and a second region, wherein the hydrolase cleavage site is positioned between the first region and the second region; and wherein the first region and the second region of the targeting agent blocker hybridize to first and second regions of the targeting agent complement on the second strand.

37. The oligonucleotide detection reagent of any one of claims 31 to 34, wherein the targeting agent complement, the hydrolase cleavage site and the targeting agent blocker are comprised on a single strand.

38. The oligonucleotide detection reagent of any one of claims 31 to 37, wherein the hydrolase cleavage site comprises a nucleic acid.

39. The oligonucleotide detection reagent of any one of claims 31 to 37, wherein the hydrolase cleavage site comprises a peptide.

40. The oligonucleotide detection reagent of any one of claims 31 to 37, wherein the hydrolase cleavage site comprises a polysaccharide.

41. The oligonucleotide detection reagent of claim 33, wherein the detectable label is detected by using light scattering, optical absorbance, fluorescence, chemiluminescence, electrochemiluminescence (ECL), bioluminescence, phosphorescence, radioactivity, magnetic field, or combinations thereof.

42. The oligonucleotide detection reagent of claim 33, wherein the detectable label is an electrochemiluminescent label, and the detecting comprises measuring an ECL signal.

43. A kit comprising:
a. at least one oligonucleotide detection reagent, wherein the oligonucleotide detection reagent comprises:
i. a targeting agent complement;
ii. a targeting agent blocker that is complementary to at least a portion of the targeting agent complement; and
iii. a hydrolase cleavage site; and
b. one or more detecting reagents for detecting an unblocked oligonucleotide.

44. The kit of claim 43, wherein the oligonucleotide detection reagent further comprises iv. a detectable label.

45. The kit of claim 43 or 44, wherein the hydrolase cleavage site comprises a nucleic acid.

46. The kit of claim 43 or 44, wherein the hydrolase cleavage site comprises a polypeptide.

47. The kit of claim 43 or 44, wherein the hydrolase cleavage site comprises a polysaccharide.

48. The kit of any one of claims 43 to 47, further comprising c) a detection surface comprising a targeting agent, wherein the targeting agent is a binding partner of the targeting agent complement, wherein uncleaved oligonucleotide detection reagent does not substantially bind to the detection surface.

49. The kit of any one of claims 43 to 47, wherein the detecting reagents comprise a targeting agent, wherein the targeting agent is a binding partner of the targeting agent complement.

50. The kit of any one of claims 43 to 49, wherein the detecting reagents comprise one or more hybridization buffers.

51. The kit of any one of claims 43 to 50, wherein the detecting reagents comprise PCR extension buffers and free nucleotides.

52. The kit of any one of claims 43 to 51, wherein the kit comprises at least two, three, four, five, six, seven, eight, nine, ten or more oligonucleotide detection reagents.

53. The kit of claim 52, wherein each hydrolase cleavage site of the two or more oligonucleotide detection reagents comprises a nucleic acid, a peptide, a polysaccharide or combinations thereof.

54. A method for detecting a hydrolase enzyme in a sample, comprising:
a. contacting the sample with an oligonucleotide detection reagent, wherein the oligonucleotide detection reagent comprises:
i. a primary targeting agent complement;
ii. a secondary targeting agent complement;
iii. a hydrolase cleavage site; and
iv. a detectable label;
wherein the hydrolase enzyme cleaves the oligonucleotide detection reagent, thereby generating (i) a cleaved secondary targeting agent complement and (ii) a first cleaved oligonucleotide detection reagent comprising the primary targeting agent complement and the detectable label:
b. binding the cleaved secondary targeting agent complement, uncleaved oligonucleotide detection reagent, or both, to a binding surface comprising a secondary targeting agent that is a binding partner of the secondary targeting agent complement;
c. immobilizing the first cleaved oligonucleotide detection reagent to a detection surface comprising a primary targeting agent, wherein the primary targeting agent is a binding partner of the primary targeting agent complement; and
d. detecting the first cleaved oligonucleotide detection reagent immobilized on the detection surface, wherein the cleaved secondary targeting agent complement and the uncleaved oligonucleotide detection reagent on the binding surface are substantially undetected,
thereby detecting the hydrolase enzyme in the sample.

55. The method of claim 54, wherein the secondary targeting agent complement is attached to a first terminus of the hydrolase cleavage site, a first terminus of the primary targeting agent complement is attached to a second terminus of the hydrolase cleavage site, and the detectable label is attached to a second terminus of the primary targeting agent complement.

56. The method of claim 54 or 55, wherein the hydrolase enzyme is a nuclease and the hydrolase cleavage site comprises a nucleic acid that is cleaved by the nuclease.

57. The method of claim 54 or 55, wherein the hydrolase enzyme is a peptidase and the hydrolase cleavage site comprises a peptide that is cleaved by the peptidase.

58. The method of claim 54 or 55, wherein the hydrolase enzyme is a glycoside hydrolase and the hydrolase cleavage site comprises a polysaccharide that is cleaved by the glycoside hydrolase.

59. The method of any one of claims 54 to 58, wherein the primary targeting agent complement further comprises a nuclease-resistant nucleotide.

60. The method of claim 59, wherein the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

61. The method of any one of claims 54 to 60, wherein the sample is contacted with one or more additional copies of the oligonucleotide detection reagent.

62. The method of any one of claims 54 to 61, wherein the secondary targeting agent complement comprises biotin, and the secondary targeting agent comprises avidin or streptavidin.

63. The method of any one of claims 54 to 62, wherein each of the primary targeting agent complement and the primary targeting agent is substantially unreactive with the secondary targeting agent and secondary targeting agent complement.

64. The method of any one of claims 54 to 63, further comprising, prior to the detecting, separating the binding surface comprising the secondary targeting agent complement from the detection surface comprising the first cleaved oligonucleotide detection reagent.

65. The method of any one of claims 54 to 64, wherein the primary targeting agent complement and the primary targeting agent comprise complementary oligonucleotides, and the immobilizing comprises hybridizing the primary targeting agent complement of the first cleaved oligonucleotide detection reagent to the primary targeting agent on the detection surface.

66. The method of any one of claims 54 to 65, wherein the detectable label is detected by using light scattering, optical absorbance, fluorescence, chemiluminescence, electrochemiluminescence (ECL), bioluminescence, phosphorescence, radioactivity, magnetic field, or combinations thereof.

67. The method of claim 66, wherein the detectable label is an electrochemiluminescent label, and the detecting comprises measuring an ECL signal.

68. The method of any one of claims 54 to 67, wherein the detection surface comprises a particle.

69. The method of any one of claims 54 to 67, wherein the detection surface comprises a well of a multi-well plate.

70. The method of any one of claims 54 to 69, wherein the detection surface comprises an electrode.

71. The method of any one of claims 54 to 70, wherein the binding surface is a particle.

72. The method of any one of claims 54 to 71, further comprising, prior to step (c), separating the binding surface from the first cleaved oligonucleotide detection reagent.

73. The method of any one of claims 54 to 72, wherein the method is a multiplexed method for detecting multiple hydrolase enzymes in a sample,
wherein step (a) comprises contacting the sample with a plurality of hydrolase enzymes and a plurality of oligonucleotide detection reagents, wherein each oligonucleotide detection reagent comprises a primary targeting agent complement, a secondary targeting agent complement, a hydrolase cleavage site, and a detectable label,
wherein, each unique hydrolase enzyme cleaves an oligonucleotide detection reagent comprising a unique hydrolase cleavage site for the hydrolase enzyme to generate (I) a cleaved secondary targeting agent complement and (II) a first cleaved oligonucleotide detection reagent comprising a unique primary targeting agent complement,
thereby generating (i) a plurality of secondary targeting agent complements and (ii) a plurality of first cleaved oligonucleotide detection reagents, wherein each first cleaved oligonucleotide detection reagent comprises a unique primary targeting agent complement:
wherein step (b) comprises binding the plurality of secondary targeting agent complements, uncleaved oligonucleotide detection reagent, or both, to a binding surface comprising a plurality of secondary targeting agents;
wherein step (c) comprises immobilizing the plurality of first cleaved oligonucleotide detection reagents to a detection surface comprising a plurality of primary targeting agents, wherein each primary targeting agent is a binding partner of a unique primary targeting agent complement; and
wherein step (d) comprises detecting the plurality of first cleaved oligonucleotide detection reagents bound to the detection surface, wherein the plurality of secondary targeting agent complements and the uncleaved oligonucleotide detection reagent on the binding surface are substantially undetected,
thereby detecting the multiple hydrolase enzymes in the sample.

74. The method of claim 73, wherein each unique primary targeting agent complement comprises a unique oligonucleotide sequence.

75. The method of claim 73 or 74, wherein each hydrolase enzyme is a nuclease and the hydrolase cleavage site of each oligonucleotide detection reagent comprises a nucleic acid.

76. The method of claim 73 or 74, wherein each hydrolase enzyme is a peptidase and the hydrolase cleavage site of each oligonucleotide detection reagent comprises a peptide.

77. The method of claim 73 or 74, wherein each hydrolase enzyme is a glycoside hydrolase and the hydrolase cleavage site of each oligonucleotide detection reagent comprises a polysaccharide.

78. The method of claim 73 or 74, wherein the sample comprises at least two hydrolases, and wherein the hydrolases are a nuclease, a peptidase, a glycoside hydrolase or combinations thereof.

79. An oligonucleotide detection reagent comprising:
i. a primary targeting agent complement;
ii. a secondary targeting agent complement;
iii. a hydrolase cleavage site; and
iv. a detectable label.

80. The oligonucleotide detection reagent of claim 79, wherein the secondary targeting agent complement is attached to a first terminus of the hydrolase cleavage site, a first terminus of the primary targeting agent complement is attached to a second terminus of the hydrolase cleavage site, and the detectable label is attached to a second terminus of the primary targeting agent complement.

81. The oligonucleotide detection reagent of claim 79 or 80, wherein the hydrolase cleavage site comprises a nucleic acid.

82. The oligonucleotide detection reagent of claim 79 or 80, wherein the hydrolase cleavage site comprises a peptide.

83. The oligonucleotide detection reagent of claim 79 or 80, wherein the hydrolase cleavage site comprises a polysaccharide.

84. The oligonucleotide detection reagent of any one of claims 79 to 83, wherein the detectable label is detected by using light scattering, optical absorbance, fluorescence, chemiluminescence, electrochemiluminescence (ECL), bioluminescence, phosphorescence, radioactivity, magnetic field, or combinations thereof.

85. The oligonucleotide detection reagent of any one of claims 79 to 83, wherein the detectable label is an electrochemiluminescent label.

86. A kit comprising:
a. at least one oligonucleotide detection reagent, wherein the oligonucleotide detection reagent comprises:
i. a primary targeting agent complement;
ii. a secondary targeting agent complement;
iii. a hydrolase cleavage site; and
iv. a detectable label; and
b. one or more detecting reagents for detecting a primary targeting agent complement.

87. The kit of claim 86, wherein the hydrolase cleavage site comprises a nucleic acid.

88. The kit of claim 86, wherein the hydrolase cleavage site comprises a polypeptide.

89. The kit of claim 86, wherein the hydrolase cleavage site comprises a polysaccharide.

90. The kit of any one of claims 86 to 89, further comprising c) a detection surface comprising a primary targeting agent, wherein the primary targeting agent is a binding partner of the primary targeting agent complement, wherein uncleaved oligonucleotide detection reagent does not substantially bind to the detection surface.

91. The kit of any one of claims 86 to 90, wherein the detecting reagents comprise a primary targeting agent, wherein the primary targeting agent is a binding partner of the primary targeting agent complement.

92. The kit of any one of claims 86 to 91, wherein the detecting reagents comprise a secondary targeting agent, wherein the secondary targeting agent is a binding partner of the secondary targeting agent complement.

93. The kit of claim 92, wherein the secondary targeting agent complement is biotin and the secondary targeting agent is avidin or streptavidin.

94. The kit of any one of claims 86 to 93, wherein the detecting reagents comprise one or more hybridization buffers.

95. The kit of any one of claims 86 to 94, wherein the kit comprises at least two, three, four, five, six, seven, eight, nine, ten or more oligonucleotide detection reagents.

96. The kit of claim 95, wherein each hydrolase cleavage site of the two or more oligonucleotide detection reagents comprises a nucleic acid, a peptide, a polysaccharide or combinations thereof.


SEQUENCE LISTING (17 sequences)

SEQ ID NO: 1 - DNA, 50 bp, Artificial Sequence
  Oligonucleotide detection reagent
atcatgtctgggttacuuuuuuuuuuactggtaacccagacatgatcggt

SEQ ID NO: 2 - DNA, 43 bp, Artificial Sequence
  Oligonucleotide detection reagent
catgtctgggttacuuuuuactggtaacccagacatgatcggt

SEQ ID NO: 3 - DNA, 43 bp, Artificial Sequence
  Oligonucleotide detection reagent
tcatgtctgggttauuuuuactggtaacccagacatgatcggt

SEQ ID NO: 4 - DNA, 48 bp, Artificial Sequence
  Oligonucleotide detection reagent
tcatgtctgggttauuuuuuuuuuactggtaacccagacatgatcggt

SEQ ID NO: 5 - DNA, 45 bp, Artificial Sequence
  Oligonucleotide detection reagent
tcatgtctgggttauguguguactggtaacccagacatgatcggt

SEQ ID NO: 6 - protein, 8 aa, Artificial Sequence
  Protease target sequence
  MOD_RES (3)..(3): Xaa is Abu
YYXNEPYK

SEQ ID NO: 7 - protein, 8 aa, Artificial Sequence
  Protease target site
GDEVDGVK

SEQ ID NO: 8 - protein, 8 aa, Artificial Sequence
  Protease target site
GWEHDGVK

SEQ ID NO: 9 - protein, 15 aa, Artificial Sequence
  Protease target site
GWEHDGVGWEHDGVK

SEQ ID NO: 10 - protein, 8 aa, Artificial Sequence
  Protease target site
GVEHDGVK

SEQ ID NO: 11 - protein, 15 aa, Artificial Sequence
  Protease target site
GVEHDGVGVEHDGVK

SEQ ID NO: 12 - protein, 5 aa, Artificial Sequence
  Protease target site
IEGRK

SEQ ID NO: 13 - protein, 5 aa, Artificial Sequence
  Protease target site
IDGRK

SEQ ID NO: 14 - DNA, 24 bp, Artificial Sequence
  Targeting agent complement sequence
actggtaacccagacatgatcggt

SEQ ID NO: 15 - DNA, 24 bp, Artificial Sequence
  Targeting agent complement sequence
ctaatagctcctgtgccctcgtat

SEQ ID NO: 16 - DNA, 24 bp, Artificial Sequence
  Targeting agent complement sequence
aatccgtcgactagcctgagaatt

SEQ ID NO: 17 - DNA, 24 bp, Artificial Sequence
  Targeting agent complement sequence
cgtaccattgaatctggagacctt